(19) United States
(12) Patent Application Publication
(10) Pub. No.: US 2011/0118587 A1
(43) Pub. Date: May 19, 2011

(54) OPTIMIZATION OF RF TRANSMIT AND GRADIENT MAGNETIC FIELD IMAGING USING RADIO FREQUENCY AND GRADIENT COILS

(76) Inventors: Peter B. Roemer, North Andover, MA (US); Richard P. Mallozzi, Middletown, MA (US); Yuan Cheng, Andover, MA (US)

(21) Appl. No.: 12/920,690

(22) PCT Filed: Mar. 4, 2009
(86) PCT No.: PCT/US09/01385

§ 371 Date: Jan. 24, 2011

Related U.S. Application Data
(60) Provisional application No. 61/033,467, filed on Mar. 4, 2008.

(51) Int. Cl. A61B 5/055

(52) U.S. Cl. 600/411

(57) ABSTRACT

The invention provides various systems, machine readable programs and methods for performing imaging using a MR scanner. The MR scanner includes at least one local radio-frequency transmit coil and at least one local gradient coil. The local radio-frequency transmit coil(s) and local gradient coil(s) cooperate to define an imaging volume. The MR scanner further includes a control system for performing an imaging operation on a patient's anatomy disposed within the imaging volume. The control system permits a selective simultaneous increase of the gradient magnetic field strength and peak B1 field strength by substantially the same factor, f.

### CROSS-REFERENCE TO RELATED APPLICATIONS

The present patent application claims the benefit of priority to U.S. Patent Application Ser. No. 61/033,467 filed Mar. 4, 2008, which is incorporated by reference herein in its entirety.

### BACKGROUND OF THE INVENTION

1. Field of the Invention

The present invention relates to improved techniques and devices for performing magnetic resonance imaging (“MRI”).

2. Description of Related Art

The vast majority of MRI systems available today are general-purpose whole body systems with built in RF body transmit and body gradient coils. There is another more limited class of dedicated extremity systems [1,2] where the main focus has been lower cost of the system and making it easier to locate.

Relative to whole body systems the trend toward higher performance solutions centers around (1) increasing the field strength for higher signal-to-noise, (2) making arrays of a large number of relatively smaller receive only RF coils [3,4] for higher signal-to-noise and for faster imaging [5], and [3] design of very high power gradient drivers capable of outputting thousands of volts and hundreds of amperes, which drive a whole body gradient coil to larger gradient fields with faster slew rates to improve imaging performance.

There are several important fundamental limitations that restrict the push to faster, stronger gradient fields and stronger RF fields. In addition to the numerous technical challenges, there are physiological limitations preventing one from crossing certain thresholds of gradient and RF field parameters.

For RF transmit coils, depending on the imaging sequence parameters, the physiological limitation is typically the amount of heat deposited in the patient, on both a local level and a whole body level. This dissipation of this heat is quantified in a parameter called Specific Absorption Rate (SAR), which is typically measured in Watts/Kg. The FDA and International regulations place different limitations on the whole body and anatomy specific (local) regions of the body.

For gradient fields, the physiological limitation is typically the induced Peripheral Nerve Stimulation (PNS). Magnetic fields that are changing in time create an associated electrical field. For typical MRI gradient fields, the induced electrical field is at frequencies in the range of 0 to 20 KHz. At high levels, this induced electrical field stimulates nerves, leading to a sensation in the patient. The amount of nerve stimulations increases with gradient slew rate and gradient amplitude. At even higher levels it could lead to dangerous stimulations. Nerve stimulation to the point of a given subject's pain threshold is generally considered safe, but practical aspects of patient comfort generally dictate lower levels of nerve stimulation.

Most whole-body MRI systems have turned to the techniques of parallel imaging [5] to overcome limitations in gradient and RF field exposure. In these techniques, large multi-channel receive arrays are used. Unlike conventional imaging, in which all spatial encoding is done with gradient fields, parallel imaging techniques use the different coils in the array to help encode the spatial information. These techniques enable coverage of k-space with fewer gradient pulses than in conventional imaging, which helps reduce exposure of the patient to the electromagnetic fields. The tradeoff is signal-to-noise: although one can acquire the same image with less exposure of the patient to gradient and RF fields, the signal-to-noise is reduced at least as fast as the square root of the acceleration factor in parallel imaging. The acceleration factor is generally considered to be the fractional increase in spatial information obtained using the parallel imaging techniques divided by the spatial information that would be acquired in the same scan time without parallel imaging.

It has been recognized that smaller local gradient coils can increase gradient performance but practical aspects has prevented their use on whole body systems. For example, a localized gradient coil is typically heavier than an RF coil, requiring specialized lifting equipment for the operator. Water cooling attachments etc all need to be flexible, reliable and safe. There are concerns about mechanical and electrical safety for a gradient coil that might be “loosely placed” on the table of a whole body system near the anatomy of interest. There are potentially large translational and rotational forces placed on a coil between the gradient coil winding and the main magnetic field should an internal short circuit occur in the wrong place of the coil.

There are examples of localized RF transmit coils such as for head imaging and imaging of the knee. In these cases the coils are typically quadrature (also called “circularly-polarized”) volume transmit and receive coils. The more recent technology of multiple surface coils is tending to replace these coils with an array of localized coils, using the body coil only for transmit.

Dedicated extremity systems started with low cost and low field permanent magnet systems [1] have now trended to higher field superconducting systems at 1.0T [2]. The manufacturers of these systems have not exploited the higher physiological limits allowed on smaller systems and taken full advantage of the associated performance improvements potentially obtained by imaging only a limited anatomical portion of the body.

### SUMMARY OF THE INVENTION

The purpose and advantages of the present invention will be set forth in and become apparent from the description that follows. Additional advantages of the invention will be realized and attained by the methods and systems particularly pointed out in the written description hereof, as well as from the appended drawings.

This disclosure describes techniques to improve system performance (and therefore image quality) in MRI scanners by use of localized gradient and RF coils to simultaneously increase a variety of variables, including the gradient magnetic field strength, slew rate, and the radio frequency (RF) field strength for smaller anatomies to obtain performance beyond what is possible in a conventional whole body system. Using an estimate for physiological exposure to gradient and RF fields, pulse sequence parameters may be adjusted in a way that is specific to the anatomy being imaged and the hardware configuration during the exam, enabling the scanner to operate at the maximum possible performance levels but still below the limits of physiological exposure.

In accordance with one aspect of the disclosure, a system for performing imaging is provided including a MR scanner. The MR scanner includes at least one local radio-frequency transmit coil and at least one local gradient coil. The local radio-frequency transmit coil(s) and local gradient coil(s) cooperate to define an imaging volume. The MR scanner further includes a control system for performing an imaging operation on a patient's anatomy disposed within the imaging volume. The control system permits a selective simultaneous increase of the gradient magnetic field strength and peak B₁ field strength by substantially the same factor, f.

In accordance with further aspects of the disclosure, the control system may be configured to reduce the duration of the B₁ transmit pulse by the factor, f when the control system increases the gradient magnetic field strength and peak B₁ field strength by the factor, f. In accordance with a further aspect, the frequency response and the spatial response of the excited spins of the region being imaged is substantially preserved as a result of simultaneously increasing the gradient magnetic field strength and peak B₁ field strength in substantially the same proportion. Moreover, the flip angle resulting from the transmit pulse is preferably substantially unchanged when increasing the gradient and peak B₁ field strength. The excitation properties of the combined RF transmit and gradient pulse are also preferably preserved when increasing the gradient and peak B₁ field strength.

In accordance with still a further aspect of the disclosure, the control system may be adapted and configured to increase the gradient strength in excess of 30 mT/m, and/or to increase the gradient strength at a rate in excess of 100 T/m/sec. In accordance with one aspect, the control system may be adapted and configured to create an estimate of a physiological limit of a region to be imaged. The estimate may be based at least in part on a predetermined value and/or on the weight of a patient. The estimate may further be based at least in part on a predetermined value established for the particular anatomy being imaged. In accordance with a related aspect, an estimate of the mass to be imaged may be created based on one or more factors selected from the group consisting of (i) the weight of the patient, (ii) the anatomy being imaged, and (iii) the size of the imaging coil. Preferably, the factor f is maximized for a given physiological limit. The physiological limit may relate to SAR arising from application of the B₁ field. In one embodiment, the B₁ field may be maximized to not exceed a limit of 20 Watts/kg. In accordance with a related aspect, the physiological limit may relate to peripheral nerve stimulation arising from electric fields induced in the patient's anatomy.

In accordance with still a further aspect of the invention, the control system may be adapted and configured to increase the ramp rate of the gradient field. In another embodiment, the control system may be adapted and configured to reduce the duration of the echo train. In accordance with one example, the echo train can be reduced by about 25 percent by the control system. In accordance with another embodiment, the imaging sequence may be optimized for imaging tissues with relatively short T2 values. For example, the imaging sequence may be optimized for cartilage.

In accordance with yet another aspect of the disclosure, the control system may be adapted and configured to selectively increase the duration of the data acquisition window for an echo train of a predetermined duration in cooperation with simultaneously increasing the gradient magnetic field strength and peak B₁ field strength. In accordance with another aspect, the gradient field strength may be selectively increased by the control system to a magnitude between about 50 mT/m and about 100 mT/m. In accordance with another aspect, the duration of the B₁ pulse may be decreased to between about 0.5 msec and about 1.0 msec. In accordance with one example, the magnitude of the maximum B₁ field strength is more than about 0.4 gauss. In accordance with another example, the signal to noise ratio increases by about 25% as a result of simultaneously increasing the gradient magnetic field strength and peak B₁ field strength.

In accordance with another embodiment, the disclosure provides a system for performing magnetic resonance imaging. The system includes a MR scanner including at least one local radio-frequency transmit coil and at least one local gradient coil that cooperate to define an imaging volume. The system further includes a control system for performing an imaging operation on a portion of a patient's anatomy disposed within the imaging volume using the MR scanner. The gradient magnetic field strength and peak B₁ field strength are dynamically adjusted by the control system in response to the specific anatomy being imaged of a particular patient.

In accordance with a further aspect of this embodiment, the MR scanner may include a plurality of local radio-frequency coils that may be selected to perform the imaging operation. The gradient magnetic field strength and peak B₁ field strength of each radio-frequency coil are dynamically adjusted in response to the specific local radio-frequency coil that is being used. If desired, the gradient magnetic field strength and peak B₁ field strength of each radio-frequency coil may be independently adjusted by the control system. Moreover, the gradient magnetic field strength and peak B₁ field strength may be adjusted in response to the particular anatomy being imaged to substantially maximize the B₁ field strength for the anatomy being imaged but remain below SAR deposition limits. If desired, the gradient magnetic field strength and peak B₁ field strength may be adjusted in response to the type of anatomy being imaged. In accordance with another aspect, the gradient magnetic field strength and peak B₁ field strength may be adjusted based on predetermined criteria. In accordance with one embodiment, the predetermined criteria may include estimates based on a pre-characterized anatomical model.

In accordance with another aspect, the disclosure provides a system for improving MR image quality comprising a MR scanner having at least one local transmit coil, at least one local gradient coil and a control system adapted and configured to control the system. The control system includes means for selectively increasing the peak B transmit field amplitude, increasing the gradient magnetic field and reducing the duration of the transmit pulse.

In accordance with a further aspect of this embodiment, the duration of the transmit pulse may be reduced by the control system to provide additional time during a pulse sequence to receive signal from a region of interest. The duration of the transmit pulse may be reduced to compress the echo train in order to acquire image data at a relatively faster rate and reduce blurring artifacts. In accordance with another aspect, the ratio of the transmit pulse duration to the period of the transmit pulse is less than about 20%, 15%, 10% or even 5%.

The disclosure further provides a system for improving MR image quality comprising a MR scanner having at least one local transmit coil, at least one local gradient coil and a control system adapted and configured to control the system. The control system is adapted and configured to selectively increase the gradient magnetic field to reduce the duration of the echo train.

The disclosure still further provides a system for improving MR image quality comprising a MR scanner having at least one local transmit coil, at least one local gradient coil and a control system adapted and configured to control the system. The control system is adapted and configured to selectively increase the peak B₁ transmit field amplitude to reduce the duration of the echo train.

The disclosure yet further provides a system for improving MR image quality comprising a MR scanner having at least one local transmit coil, at least one local gradient coil and a control system adapted and configured to control the system. The control system is adapted and configured to perform an imaging operation on a region of interest using the local coil, wherein the peak B₁ field strength is dynamically adjusted by the control system in response to the specific anatomy being imaged of a particular patient.

In accordance with a further aspect of this embodiment, the peak B₁ field strength is set by the control system before the scan is initiated. This can be influenced by the input of an operator. In accordance with a further aspect, the MR scanner may include a plurality of local radio-frequency coils that may be selected to perform the imaging operation. The peak B₁ field strength may be dynamically adjusted by the control system in response to the specific local radio-frequency coil that is being used. The control system can be adapted energize a plurality of coils to perform the imaging operation. The gradient magnetic field strength and peak B₁ field strength may be adjusted by the control system in response to the particular anatomy being imaged to substantially maximize the B₁ field strength for the anatomy of interest but remain below SAR deposition limits. The peak B₁ transmit field of the imaging coil can be configured by the control system to be in excess of 50 μT. In accordance with one embodiment, the peak B₁ field strength may be dynamically adjusted by the control system to provide SAR deposition in the region of interest substantially in excess of 4 Watts/kg.

In accordance with a further aspect, any of the MR scanners disclosed herein may be specially adapted and configured for imaging extremities of a patient. For example, the MR scanner may include an imaging bore having a maximum transverse dimension that is less than about 50, 40, 30, 20 or 10 centimeters.

The disclosure also provides a system for performing imaging including a MR scanner. The scanner includes a main magnet adapted and configured to apply a static magnetic field and at least one local radio-frequency transmit coil defining an imaging volume, wherein the ratio of the transmit field to the background field B₁/B₀ is more than about 5×10⁻⁵ when performing an imaging operation on the patient's anatomy. It will be appreciated that this embodiment can be modified and adjusted to include any suitable modifications disclosed and taught herein.

The disclosure further provides a machine readable program disposed on a computer readable medium containing instructions for controlling a system for performing imaging comprising a MR scanner, the MR scanner including a control system, at least one local radio-frequency transmit coil and at least one local gradient coil, wherein the at least one local radio-frequency transmit coil and at least one local gradient coil cooperate to define an imaging volume. The program includes means for controlling the system to perform an imaging operation on a patient's anatomy disposed within the imaging volume, wherein the program permits a selective simultaneous increase of the gradient magnetic field strength and peak B₁ field strength by substantially the same factor, f.

In accordance with a further aspect, the program may reduce the duration of the B₁ transmit pulse by the factor, f when the program increases the gradient magnetic field strength and peak B₁ field strength by the factor, f. The frequency response and the spatial response of the excited spins of the region being imaged is substantially preserved as a result of the program acting simultaneously increase the gradient magnetic field strength and peak B₁ field strength in substantially the same proportion. The flip angle resulting from the transmit pulse is preferably substantially unchanged when increasing the gradient and peak B₁ field strength. The excitation properties of the combined RF transmit and gradient pulse are also preferably preserved when increasing the gradient and peak B₁ field strength.

In accordance with a further aspect, the program may be adapted and configured to permit the selective increase the gradient strength in excess of 30 mT/m. If desired, the program may permit the gradient strength to be selectively increased at a rate in excess of 100 T/m/sec. In accordance with still a further aspect, the program may be adapted and configured to create an estimate of a physiological limit of a region to be imaged based on user defined input. The estimate may be based at least in part on a predetermined value, and/or the weight of a patient. The estimate may be based at least in part on a predetermined value established for the particular anatomy being imaged. If desired, an estimate of the mass to be imaged may be created based on one or more factors selected from the group consisting of (i) the weight of the patient, (ii) the anatomy being imaged, and (iii) the size of the imaging coil. Preferably, the program maximizes the factor, f, for a predetermined physiological limit. The physiological limit may relate to SAR arising from application of the B₁ field of the system. The B₁ field may be established by the program to not exceed a limit of 20 Watts/kg. The physiological limit may relate to peripheral nerve stimulation arising from electric fields induced in the patient's anatomy.

In accordance with a further aspect the program may further be adapted and configured to increase the ramp rate of the gradient field. The program may be adapted and configured to selectively reduce the duration of the echo train when increasing the magnitude of the B₁ transmit field and gradient field. For example, the echo train may be reduced by about 25 percent. The imaging sequence may be optimized by the program for imaging tissues with relatively short T2 values, such as cartilage. In accordance with a further aspect, the program may be adapted and configured to selectively increase the duration of the data acquisition window for an echo train of a predetermined duration in cooperation with simultaneously increasing the gradient magnetic field strength and peak B₁ field strength. The gradient field strength may be selectively increased by the program to a magnitude between about 5 G/cm and about 10 G/cm. The duration of the B₁ pulse may be decreased by the program to between about 0.5 msec and about 1.0 msec. In accordance with one embodiment, the program may be adapted and configured to permit the magnitude of the maximum B₁ field strength to be more than about 0.4 gauss. In accordance with a further embodiment, the signal to noise ratio may be increased by about 25% as a result of the program simultaneously increasing the gradient magnetic field strength and peak B₁ field strength.

In further accordance with the disclosure, a machine readable program disposed on a computer readable medium containing instructions for controlling a system for performing imaging comprising a MR scanner is provided. The MR scanner includes a control system, at least one local radio-frequency transmit coil and at least one local gradient coil, wherein the at least one local radio-frequency transmit coil and at least one local gradient coil cooperate to define an imaging volume. The program includes means for dynamically adjusting the gradient magnetic field strength and peak B₁ field strength in response to the specific anatomy being imaged of a particular patient.

In further accordance with this aspect of the disclosed embodiments, the MR scanner includes a plurality of local radio-frequency coils that may be selected to perform the imaging operation, and the gradient magnetic field strength and peak B₁ field strength of each radio-frequency coil are dynamically adjusted by the program in response to the specific local radio-frequency coil that is being used. The gradient magnetic field strength and peak B₁ field strength of each radio-frequency coil may be adjusted independently by the program. The gradient magnetic field strength and peak B₁ field strength may be adjusted by the program in response to the particular anatomy being imaged to substantially maximize the B₁ field strength for the anatomy being imaged but remain below SAR deposition limits. The gradient magnetic field strength and peak B₁ field strength may be adjusted by the program in response to the type of anatomy being imaged. The gradient magnetic field strength and peak B₁ field strength may be adjusted by the program based on predetermined criteria. The predetermined criteria may include estimates based on a pre-characterized anatomical model in a file that can be referenced by the program.

In further accordance with the disclosed embodiments, a machine readable program is provided, disposed on a computer readable medium containing instructions for controlling a system for performing imaging comprising a MR scanner. The MR scanner includes a control system, at least one local radio-frequency transmit coil and at least one local gradient coil, wherein the at least one local radio-frequency transmit coil and at least one local gradient coil cooperate to define an imaging volume. The program includes means for selectively increasing the peak B₁ transmit field amplitude, increasing the gradient magnetic field and reducing the duration of the transmit pulse.

In further accordance with this aspect of the disclosed embodiments, the duration of the transmit pulse may be reduced by the program to provide additional time during a pulse sequence to receive signal from a region of interest. The duration of the transmit pulse may be reduced by the program to compress the echo train in order to acquire image data at a relatively faster rate and reduce blurring artifacts. In accordance with a further aspect, the ratio of the transmit pulse duration to the period of the transmit pulse may be less than about 20%, 15%, 10%, or even 5%.

The present disclosure also provides a machine readable program disposed on a computer readable medium containing instructions for controlling a system for performing imaging including a MR scanner. The MR scanner includes a control system, at least one local radio-frequency transmit coil and at least one local gradient coil, wherein the at least one local radio-frequency transmit coil and at least one local gradient coil cooperate to define an imaging volume. The program includes means for selectively increasing the gradient magnetic field to reduce the duration of the echo train.

The present disclosure also provides a machine readable program disposed on a computer readable medium containing instructions for controlling a system for performing imaging including a MR scanner. The MR scanner includes a control system, at least one local radio-frequency transmit coil and at least one local gradient coil, wherein the at least one local radio-frequency transmit coil and at least one local gradient coil cooperate to define an imaging volume. The program includes means for increasing the peak B₁ transmit field amplitude to reduce the duration of the echo train.

The present disclosure also provides a machine readable program disposed on a computer readable medium containing instructions for controlling a system for performing imaging including a MR scanner. The MR scanner includes a control system, at least one local radio-frequency transmit coil and at least one local gradient coil, wherein the at least one local radio-frequency transmit coil and at least one local gradient coil cooperate to define an imaging volume. The program includes means for performing an imaging operation on a region of interest using the local coil, wherein the peak B₁ field strength is dynamically adjusted by the program in response to the specific anatomy being imaged of a particular patient.

In further accordance with this aspect of the disclosed embodiments, the peak B₁ field strength may be set by the program before the scan is initiated. The MR scanner may include a plurality of local radio-frequency coils that may be selected to perform the imaging operation, and the peak B₁ field strength may be dynamically adjusted by the program in response to the specific local radio-frequency coil that is being used. If desired, the program can energize a plurality of coils to perform the imaging operation. Also, the gradient magnetic field strength and peak B₁ field strength may be adjusted by the control system in response to the particular anatomy being imaged to substantially maximize the B₁ field strength for the anatomy of interest but remain below SAR deposition limits. The peak B transmit field of the imaging coil can be configured by the program to be in excess of 50 μT. If desired, the peak B₁ field strength may be dynamically adjusted by the program to provide SAR deposition in the region of interest substantially in excess of 4 Watts/kg. In accordance with a further aspect, the machine readable program may be adapted for use with a system that is specially adapted and configured for imaging extremities of a patient.

The present disclosure also provides a machine readable program disposed on a computer readable medium containing instructions for controlling a system for performing imaging comprising a MR scanner. The MR scanner includes a control system, at least one local radio-frequency transmit coil and at least one local gradient coil, wherein the at least one local radio-frequency transmit coil and at least one local gradient coil cooperate to define an imaging volume, wherein the program includes means for performing an imaging operation on a region of interest, wherein the ratio of the transmit field to the background field B₁/B₀ is more than about 5×10⁻⁵.

The present disclosure also provides a method for performing imaging. The method includes providing a MR scanner including at least one local radio-frequency transmit coil and at least one local gradient coil that cooperate to define an imaging volume, and introducing a portion of a patient's anatomy into the imaging volume. The method further includes performing an imaging operation on the patient's anatomy using the MR scanner by simultaneously increasing the gradient magnetic field strength and peak B₁ field strength by substantially the same factor, f.

In further accordance with the disclosed method, the duration of the B₁ transmit pulse may be reduced by the factor, f, when the gradient magnetic field strength and peak B₁ field strength are increased by the factor, f. The frequency response and the spatial response of the excited spins of the region being imaged is substantially preserved as a result of simultaneously increasing the gradient magnetic field strength and peak B₁ field strength in substantially the same proportion. The flip angle resulting from the transmit pulse is preferable substantially unchanged when increasing the gradient and peak B₁ field strength. The excitation properties of the combined RF transmit and gradient pulse are preferably preserved when increasing the gradient and peak B₁ field strength. In accordance with one embodiment, the gradient strength is increased in excess of 30 mT/m. In accordance with another embodiment, the gradient strength is increased at a rate in excess of 100 T/m/sec.

In accordance with a further aspect of the disclosed methods, an estimate of a physiological limit of a region to be imaged can be created. The estimate may be created based at least in part on a predetermined value. The estimate may be based at least in part on the weight of a patient. If desired, the estimate may be based at least in part on a predetermined value established for the particular anatomy being imaged. By way of further example, an estimate of the mass to be imaged may be created based on one or more factors selected from the group consisting of (i) the weight of the patient, (ii) the anatomy being imaged, and (iii) the size of the imaging coil. In accordance with a preferred embodiment, the factor f is maximized for a given physiological limit. The physiological limit may relate to SAR arising from application of the B₁ field. In accordance with one embodiment, the B₁ field is maximized to not exceed a limit of 20 Watts/kg. In accordance with another embodiment, the physiological limit may relate to peripheral nerve stimulation arising from electric fields induced in the patient's anatomy.

In accordance with a further aspect of the disclosed embodiments, the method may further include increasing the ramp rate of the gradient field and/or reducing the duration of the echo train. In accordance with one example, the echo train may be reduced by about 25 percent. In accordance with another embodiment, the imaging sequence may be optimized for imaging tissues with relatively short T2 values, such as for cartilage. If desired, the method may further include increasing the duration of the data acquisition window for an echo train of a predetermined duration in cooperation with simultaneously increasing the gradient magnetic field strength and peak B₁ field strength. If desired, the gradient field strength may be increased to a magnitude between about 5 G/cm and about 10 G/cm. Moreover, the duration of the B₁ pulse is decreased to between about 0.5 msec and about 1.0 msec. By way of further example, the magnitude of the maximum B₁ field strength is more than about 0.4 gauss. In accordance with a preferred embodiment, the signal to noise ratio increases by about 25% as a result of simultaneously increasing the gradient magnetic field strength and peak B₁ field strength.

In further accordance with the preferred embodiments, a method for performing imaging is provided. The method includes providing a MR scanner including at least one local radio-frequency transmit coil and at least one local gradient coil that cooperate to define an imaging volume. The method further includes introducing a portion of a patient's anatomy into the imaging volume, and performing an imaging operation on the patient's anatomy using the MR scanner, wherein the gradient magnetic field strength and peak B₁ field strength are dynamically adjusted in response to the specific anatomy being imaged of a particular patient.

In accordance with a further aspect of the disclosed embodiments the, MR scanner may include a plurality of local radio-frequency coils that may be selected to perform the imaging operation. The gradient magnetic field strength and peak B₁ field strength of each radio-frequency coil may accordingly be dynamically adjusted in response to the specific local radio-frequency coil that is being used. The gradient magnetic field strength and peak B₁ field strength of each radio-frequency coil may be independently adjusted in response to the specific local radio-frequency coil that is being used. The gradient magnetic field strength and peak B₁ field strength may be adjusted in response to the particular anatomy being imaged to substantially maximize the B₁ field strength for the anatomy of interest but remain below SAR deposition limits. If desired, the gradient magnetic field strength and peak B₁ field strength may be adjusted in response to the type of anatomy being imaged. In accordance with one example, the gradient magnetic field strength and peak B₁ field strength may be adjusted based on predetermined criteria. For example, the predetermined criteria may include estimates based on a pre-characterized anatomical model.

The disclosed embodiments further provide a method for improving MR image quality, including increasing the peak B₁ transmit field amplitude, increasing the gradient magnetic field and reducing the duration of the transmit pulse simultaneously.

In further accordance with the disclosed embodiments, the duration of the transmit pulse may be reduced to provide additional time during a pulse sequence to receive signal from a region of interest. The duration of the transmit pulse may be reduced to compress the echo train in order to acquire image data at a relatively faster rate and reduce blurring. If desired, the ratio of the transmit pulse duration to the period of the transmit pulse is less than about 20%, 15%, 10% or even 5%.

In accordance with still a further aspect of the disclosure, a method for improving MR image quality is provided, including increasing the gradient magnetic field to reduce the duration of the transmit pulse.

In accordance with yet a further aspect of the disclosure, a method for improving MR image quality is provided, including increasing the peak B₁ transmit field amplitude to reduce the duration of the transmit pulse.

The disclosed embodiments further provide a method for performing imaging. the method includes providing a MR scanner including at least one local radio-frequency transmit coil defining an imaging volume, and introducing a portion of a patient's anatomy into the imaging volume. The method further includes performing an imaging operation on the patient's anatomy using the at least one local coil, wherein the peak B₁ field strength is dynamically adjusted in response to the specific anatomy being imaged of a particular patient.

In accordance with a further aspect of the aforementioned method, the peak B₁ field strength is selected before the scan is initiated. If desired, the MR scanner can be equipped with a plurality of local radio-frequency coils that may be selected to perform the imaging operation, and the peak B₁ field strength may be dynamically adjusted in response to the specific local radio-frequency coil that is being used. In accordance with one embodiment, the gradient magnetic field strength and peak B₁ field strength may be adjusted in response to the particular anatomy being imaged to substantially maximize the B₁ field strength for the anatomy of interest but remain below SAR deposition limits. In accordance with one example, the peak B₁ transmit field of the imaging coil is in excess of 50 μT. In accordance with another embodiment, the SAR deposition is substantially in excess of 4 Watts/kg.

The disclosed embodiments further provide a method for performing imaging. The method includes providing a MR scanner including a main magnet adapted and configured to apply a static magnetic field and at least one local radio-frequency transmit coil defining an imaging volume. The method further includes introducing a portion of a patient's anatomy into the imaging volume, and performing an imaging operation on the patient's anatomy using the local coil, wherein the ratio of the strength of the transmit field to the background field, B₁/B₀, is more than about 5×10⁻⁵.

It is to be understood that both the foregoing general description and the following detailed description are exemplary and are intended to provide further explanation of the embodiments disclosed herein. The accompanying figures herein, which are incorporated in and constitute part of this specification, are included to illustrate and provide a further understanding of the methods, systems and software programs of the invention. Together with the description, the drawings serve to explain the principles of the invention.

### BRIEF DESCRIPTION OF THE FIGURES

FIG. 1 is a depiction of readout (Gx) and slice select (Gz) gradients, in combination with RF transmit field aspects for typical imaging parameters.

FIG. 2 is a depiction of readout (Gx) and slice select (Gz) gradients, along with RF transmit field. The maximum gradient strength has been increased to 7 G/cm.

FIG. 3 is a waveform diagram for the fast spin echo sequence. The rf pulse duration is 3 msec. The slice gradient strength is 1.06 Gauss/cm and the maximum B₁ is 0.308 Gauss. The flattop portion of the readout gradient (GX) shows the length of time collecting image information, in this case 256 points at a data rate of 65 KHz.

FIG. 4 is a waveform diagram for the fast spin echo sequence. The RF pulse duration has been compressed to 0.7 msec (factor f=4.28), the maximum B₁ increased to 1.3 Gauss resulting in a shorter overall echo train. The data sample rate is 256 points at a data rate of 65 KHz, the same as FIG. 3.

FIG. 5 is a waveform diagram for the fast spin echo sequence. The RF pulse duration has been compressed to 0.7 msec (factor f=4.28), the maximum B₁ increased to 1.3 Gauss. The data sample rate is 256 points at a data rate of 42 KHz resulting in a overall duration of the echo train similar to FIG. 3 but with a 26% increase in SNR.

FIG. 6 depicts readout (Gx) and slice select (Gz) gradients, along with aspects of the RF transmit field. The maximum gradient strength has been increased to 7 G/cm.

FIG. 7 depicts a plot of average lower arm length as a function of body mass.

FIG. 8 is a plot of male and female child and adult lower arm (wrist to elbow) length as a function of body mass.

FIG. 9 is a plot of male and female hand length (wrist to finger tip) as a function of body mass.

FIG. 10 is a plot of male and female upper arm length (elbow to shoulder) as a function of body mass.

FIG. 11 is a plot of male and female foot length (tip of toes to heel) as a function of body mass.

FIG. 12 is a plot of male and female lower leg (ankle to knee) as a function of body mass.

FIG. 13 is a plot of male and female upper leg length (knee to hip) as a function of body mass.

FIG. 14 depicts a conical section of a single body segment.

FIG. 15 depicts the upper bound error for the body segment length for each age group and sex. The adult army data is lumped into the average age of 27 for purposes of this plot.

FIG. 16 depicts series resistance contribution from the limb plotted as a function of the calculated average radius of the limb near each joint. The solid line represents a best fit to the data.

FIG. 17 is a simplified block diagram of a MR scanner.

### DETAILED DESCRIPTION OF PREFERRED EMBODIMENTS

Reference will now be made in detail to the present preferred embodiments of the invention, examples of which are illustrated in the accompanying drawings. The method and corresponding steps of the invention will be described in conjunction with the detailed description of the system.

Embodiments of the subject invention relate to the use of smaller gradient and RF coils, and dynamically adjusting the gradient and RF pulse sequences in a way that depends upon the anatomy being imaged and the hardware being used during the exam, preferably for imaging only a portion of the body for purposes of enhancing imaging performance. The methods improve upon whole body systems by operating the coils at RF and gradient field strengths that would otherwise create unacceptably excessive heating or Peripheral Nerve Stimulation (PNS).

In accordance with one embodiment, the smaller geometry of the anatomy being imaged (and the scanner) leads to lower exposure to electromagnetic fields, allowing the use of stronger and shorter gradient and RF pulses. Whole body systems generally transmit to the entire torso, even if just imaging a small part of the body or receiving with a smaller coil and therefore cannot achieve gradient and RF performance levels possible in a relatively smaller hardware geometry. This limitation is generally physiological. Furthermore, in the case of extremities, higher local exposure to electromagnetic fields is permissible relative to the torso and the head.

In accordance with another embodiment, the use of anatomy, patient, and coil specific information to estimate exposure of the patient to electromagnetic fields, and then adjustment of pulse sequence parameters to operate closer to exposure limits helps facilitate optimal imaging performance. In one aspect, optimal performance depends upon the ability to use the most aggressive gradient and RF pulses that physiological limitations (described above) will allow. Every scan may be adjusted during the exam if desired, so that the system may iteratively approach the limits dictated by physiological response.

The combination of higher B₁ and gradient strength adjusted in proportion allows RF excitation and refocusing pulses to be much shorter in time while simultaneously leaving their desired frequency characteristics, such as slice definition, unchanged. This significantly reduces the amount of time used to excite or manipulate the spins and allows more time for acquisition of signal. Since higher B₁ and gradient fields can safely be obtained using this method, the image signal-to-noise and other desired quantities such as resolution and reduced blurring is improved over whole body systems.

The amount of energy is deposited in tissue is dependent on a variety of factors. For example, the amount of deposited energy depends on the intensity of the B₁ transmit magnetic field. Specifically, deposited energy is proportional to the square of the B₁ transmit field. Deposited energy also depends on the duration of the B₁ transmit pulse, as well as the shape of the pulse. Generally, deposited energy decreases as 1/(pulse duration). In addition, deposited energy also depends on the size of the object being imaged. Specifically, larger objects being imaged couple better with the transmit field because they have a larger cross sectional area. Finally, the electrical conductivity of the tissue at the frequency of interest also influences the degree to which induced current can flow in the tissue, resulting in deposited energy.

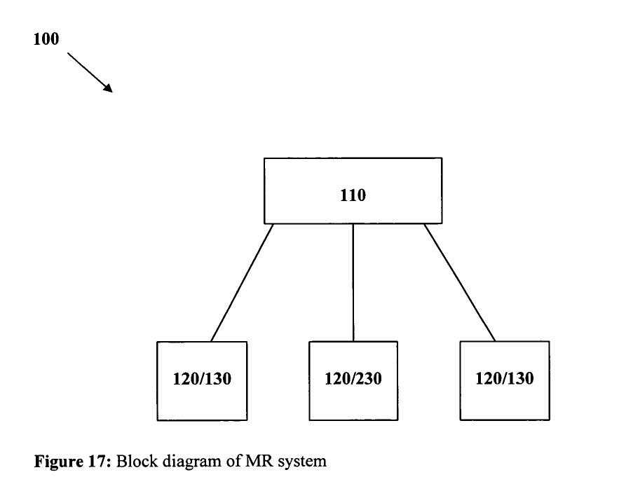

The use of a dedicated system to contain the gradient coil in a fixed geometry allows a small gradient subsystem to be safely constructed and operated at high performance without concern about mechanical forces, concern about the operator lifting of the device or concerns about the electrical safety aspects of water and power connections. FIG. 17 presents a simple block diagram of a MR scanner system 100 including a control system 110 having various control systems (e.g., computers containing appropriate software for controlling waveform generators and interface subsystems) for operating one or more RF coils 120 and/or gradient coils 130. The general theory of operation of MR scanners, RF coils and gradient field coils are well-known and need not be explained here.

As embodied herein, it is possible to move beyond gradient and RF field limitations imposed on whole-body MR scanners. In an exemplary approach, the geometry of the scanner is chosen to limit exposure of the gradient and RF fields to a smaller anatomical portion of the body. A scanner that is dedicated to a restricted region of the anatomy, such as extremities or head, can apply larger and more quickly ramped gradient fields, as well as larger RF fields before reaching the physiological limits. Although whole-body scanners can image the same anatomy, they cannot restrict the exposure to gradient and RF fields to small regions of the anatomy without special dedicated hardware that fundamentally changes the nature of the system design.

Advantages relate to both technical (engineering) ability to achieve higher gradient RF fields and ramp rates, as well as to less restrictive constraints due to physiological limitations. The physiological limitations of peripheral nerve stimulation for gradient coils and SAR deposition for RF coils scale with the size of the anatomy exposed to the fields. Furthermore, in the case of SAR deposition in the extremities, the current international safety standards [6] limit the local SAR in the to 20 Watts/Kg, whereas in the torso it is 10 Watts/Kg and whole body dose at 4 Watts/Kg. Exposing the entire body generally causes the whole body dose to be the limiting factor, causing the local SAR to operate well below it potential limits.

An MRI system dedicated to a restricted part of the anatomy can exploit technical advantages of its smaller size that enable the hardware to achieve the same performance more easily, but also exploit the advantages of the less restricted physiological limitations. This results in a number of advantages.

First, the gradient field magnitude and slew rate are usually cut off in a whole-body scanner due to peripheral nerve stimulation. If only extremities are exposed to the large gradient fields, then the peripheral nerve stimulation is significantly less than it is in a whole-body scanner, enabling the gradient system to operate at higher gradient amplitudes and ramp rates. The smaller the anatomy exposed, the higher the permitted gradient strength and slew rate.

Second, SAR deposition is limited by both the local rate of deposition and the total heat deposited in the tissue. In an analogous manner to gradient magnetic fields, exposing a smaller region of anatomy to RF fields greatly reduces the total heat deposited for the same B₁ field, enabling higher B₁ RF fields to be used in the manipulation of nuclear spins that in turn result in improved image quality.

The localized gradient and RF coils can be operated safely because of their fixed mechanical location.

### Use of Higher Gradient and RF Fields to Improve Image Quality

It is widely understood within the field of MM that higher gradient fields and ramp rates, as well as higher RF fields, can lead to improved image quality. What is not recognized is the degree to which the simultaneous improvements in both gradient performance and RF transmit field can significantly improve image quality. This improvement in image quality is explained in this section for one pulse sequence called Fast Spin Echo (FSE).

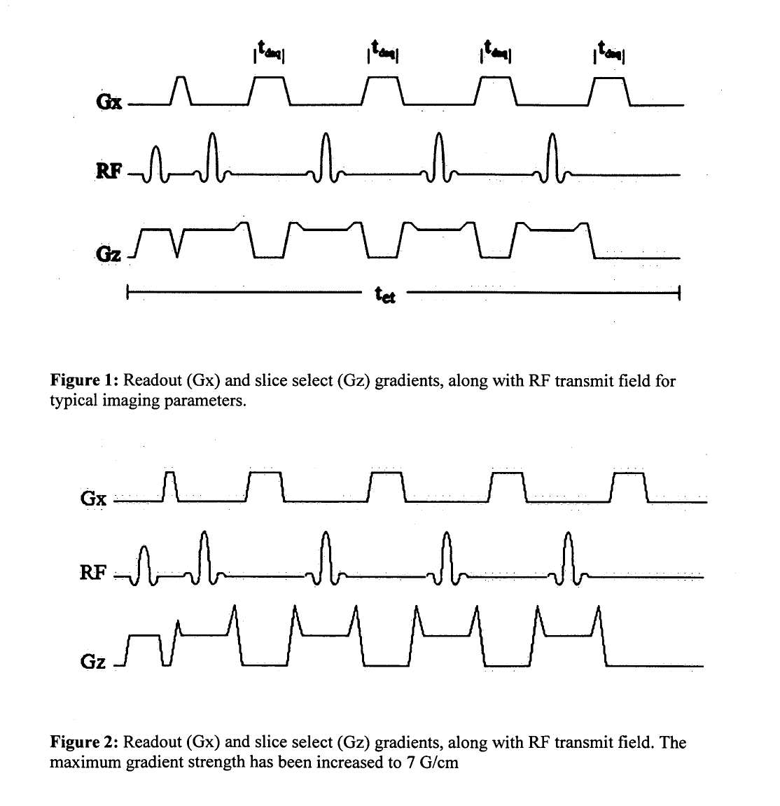

FIG. 1 is a section of an FSE pulse sequence waveform, displaying only the readout (Gx) and slice select gradients. The main pulses on the readout gradient Gx coincide with the time during which the receiver is turned on and signal is being acquired. For explanatory purposes here we start with typical pulse sequence parameters of 4 mm slice thickness, 256 readout points, Echo spacing of 16 milliseconds and data acquisition bandwidth of 50 kHz. The maximum RF field of 35 μT on the 180 deg pulse is presented with a maximum gradient strength of 1.5 G/cm.

As a figure of merit, related to the SNR and image blurring, we focus in on the total duration of the echo train, labeled tₑₜ, and t_{daq}, the duration of data acquisition window, leaving other scan parameters fixed. The total duration of the echo train is important because during this time the signal is lost due to the tissue specific T1 and T2 relaxation processes. The total time of data collection is significant as it relates to Signal-to-Noise Ratio (SNR).

The SNR per TR period is given by:

$$S N R ∝ V ( 1 - {e}^{- TR / T 1} ) {e}^{- TE / T 2} \sqrt{\frac{{N}_{etl} {N}_{read}}{BW}} \qquad ( A )$$

wherein V is the voxel volume, TR is the repetition time, TE is the echo time, BW is the receiver bandwidth in Hz, Nₑₜₗ is the number of echoes in the echo train, and N_{read} is the number of readout points in a single acquisition window. For this fast spin echo example, the echo time can be placed in the center of any one of the Nₑₜₗ echoes. Placing the echo at the last echo position gives more of a T2-weighted image. Placing the echo at a shorter time give more of a proton-density-weighted image.

According to Equation A and FIG. 1, one can see that a significant fraction of the time during a pulse sequence is dedicated to spin manipulation rather than to data acquisition. Though this spin manipulation is needed, it is only the time spent acquiring data that is actually increasing the signal-to-noise of the image, a key parameter in image quality. Any change in parameters that enables a larger fraction of the time to be spent on the data acquisition function rather than spin manipulation increases the signal-to-noise ratio. Additional signal strength is lost due to T2 decay as the data acquisition is pushed out to longer time periods.

FIG. 2 shows the impact of increasing the gradient strength from 1.5 G/cm to 7 G/cm, keeping other imaging parameters unchanged. The portion of the gradient waveform related to the actual slice selection pulse must remain the same, in order to keep the same slice profile. However, the crusher gradients can be increased resulting in an overall reduced echo spacing and echo train duration. The reduced echo spacing can be used in a variety of ways. The simplest benefit is to keep the overall echo train duration and number echoes the same, while decreasing the acquisition bandwidth (BW). Alternatively, the number of echoes can be kept the same and the SNR benefit comes by acquiring the data a shorter point in time and allowing for more image slices in the overall study.

An ingredient in achieving reduced echo spacings is the ability to run the system at higher B₁ fields. SAR limitations can often prevent use of high B₁ fields in a whole body system. In a dedicated system, the limitations on the B₁ are not as stringent. Furthermore, additional benefit can be derived by setting the limitation as it applies to the specific anatomy being imaged, rather than in a way that applies to all anatomies all the way through the worst-case (largest) anatomies. To realize this benefit, the RF transmit bandwidth, the slice gradient strength and B₁ overall amplitude are all increased by the same factor f. The duration of the RF pulse reduces by a factor of f while simultaneously preserving the details of the frequency characteristics the transmit pulse. The details of the slice profile remain unchanged.

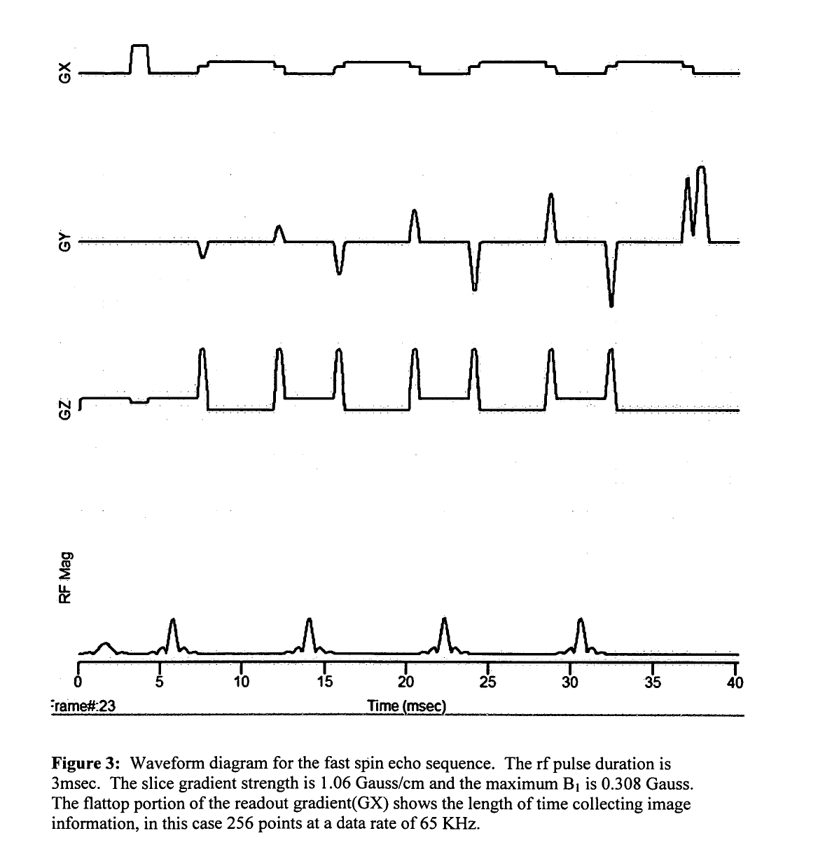

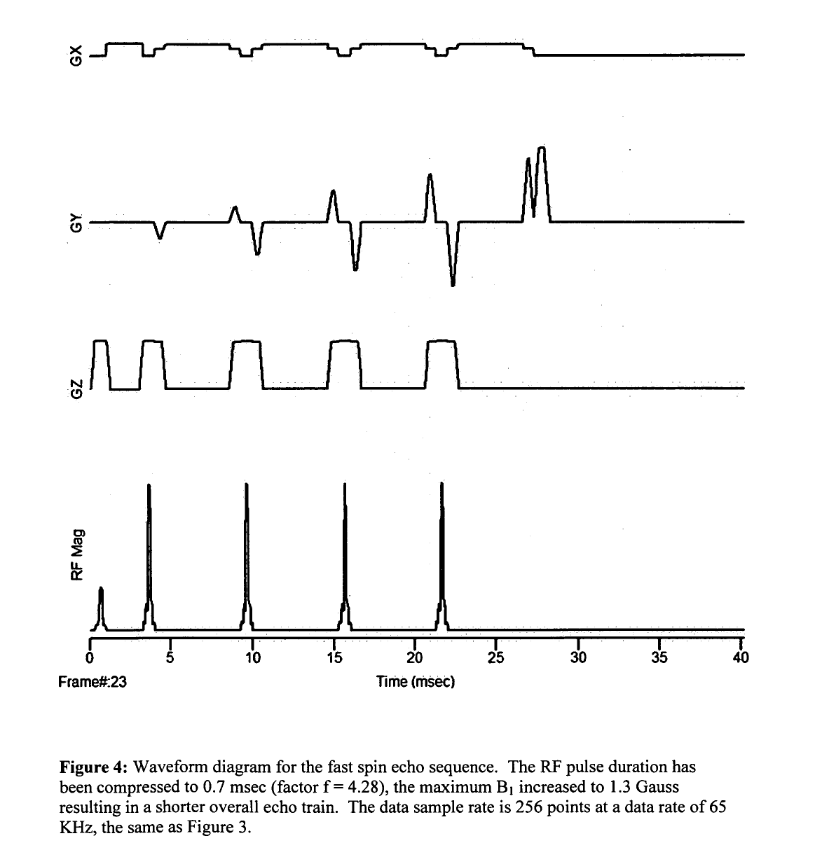

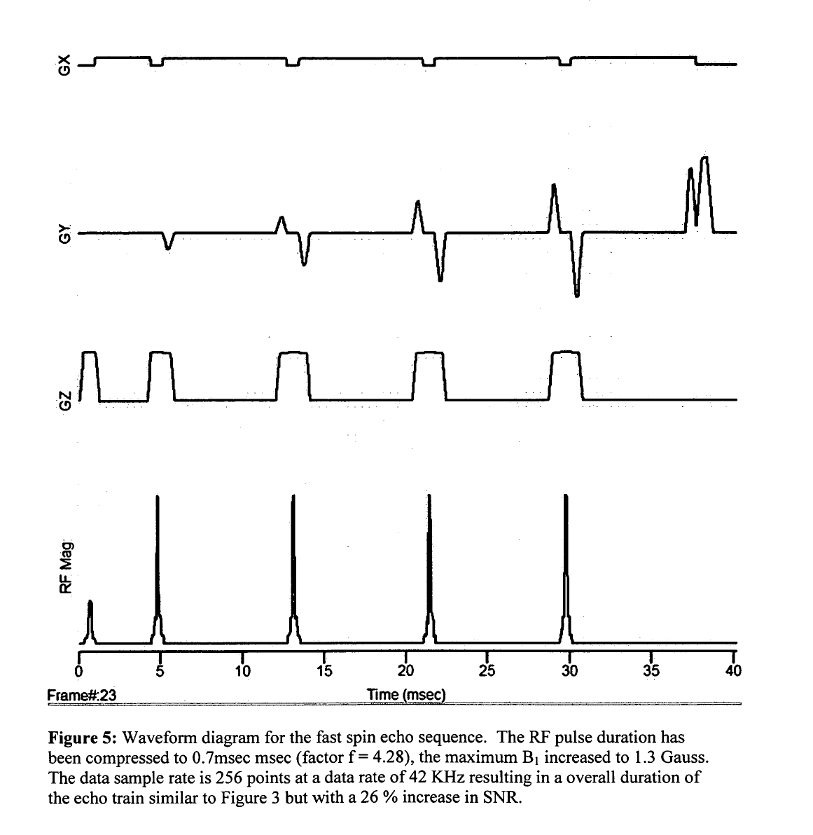

As an illustrative example of the benefit, we analyze a specific Fast Spin Echo pulse sequence and demonstrate the benefit to scan time and SNR. FIGS. 3, 4 and 5 illustrate the improvements possible with the bandwidth compression techniques. In FIG. 3, the RF pulse duration is 3 msec, typical of the duration on a whole body scanner. FIGS. 4 and 5 show the effect of pulse compression whereby the slice gradient (Gz) and the maximum B₁ (RF mag) has been increased in the same proportion. The RF pulse duration has been reduced to about 0.7 msec for a compression factor of 4.28, yet the overall slice profile and definition remain the same. In FIG. 4 the receiver bandwidth is maintained the same as in FIG. 3 (256 points with a 65 KHz sample) rate. However, the benefit of the compression has resulted in a shorter overall echo train, 30 msec versus 40 msec resulting in higher SNR for shorter T2 components, reduced blurring, and reducing scan time.

In FIG. 5 the bandwidth has been reduced to 41 KHz, and the overall echo train duration is same as in FIG. 1 (40 msec). The resultant SNR is increased by the square root of (65/41) or 26%.

In terms of the duration of the transmit pulse as compared to the echo time during the period of a transmit pulse (i.e., the time between maximum transmit intensity in consecutive pulses), typical pulse duration is about 3 milliseconds for a whole body system. Thus, for example, 2 pulses with an echo spacing of about 7 milliseconds results in ½ of each pulse to fall within the 7 millisecond period. This provides a ratio of the duration of the transmit pulse to the period of the transmit pulse of 3/7, or 42%. By way of similar example, an echo spacing of 15 milliseconds for a transmit pulse duration results in a ratio of the duration of the transmit pulse to the period of the transmit pulse of 20%. Using techniques embodied herein, it is possible to achieve a pulse duration of about 0.5 msec, resulting in a ratios of the duration of the transmit pulse to the period of the transmit pulse for 7 millisecond and 15 millisecond periods of 7.1% and 3.3%, respectively. This naturally provides more time for data acquisition. Thus, it is possible to provide a ratio of the duration of the transmit pulse to the period of the transmit pulse in an amount less than 20, 15, 10 or even 5 percent using the teachings herein.

The ability to reduce the duration of the RF pulses depends critically on the ability to raise the peak amplitude. This dependency arises directly from the Bloch Equations that describe the behavior of a magnetic moment M in a magnetic field B:

$$\frac{d \vec{M}}{d t} = γ \vec{M} \times \vec{B} \frac{d \vec{M}}{d t} = γ \vec{M} \times \vec{B}$$

Here, γ is the gyromagnetic ratio for protons in a water molecule, 42.58 MHz/Tesla. This equation also applies in a reference frame that rotates with the spins precession around the main magnetic field, where B now becomes the excess magnetic field beyond the static field (such as from gradient and RF fields). From this equation, one can see quickly the requirements to reduce the duration of the pulse by a factor f without affecting the final magnetization. Simply perform the transformation t→t/f and B→fB, and the equation remains invariant (f cancels from both sides). Thus, to shorten the duration of the pulse manipulating the spins, all excess fields (beyond the static magnetic field), including the gradient and RF fields, must become stronger by the same factor. The final magnetization will be identical to the final magnetization that occurs without compressing the pulse duration and increasing the field strengths.

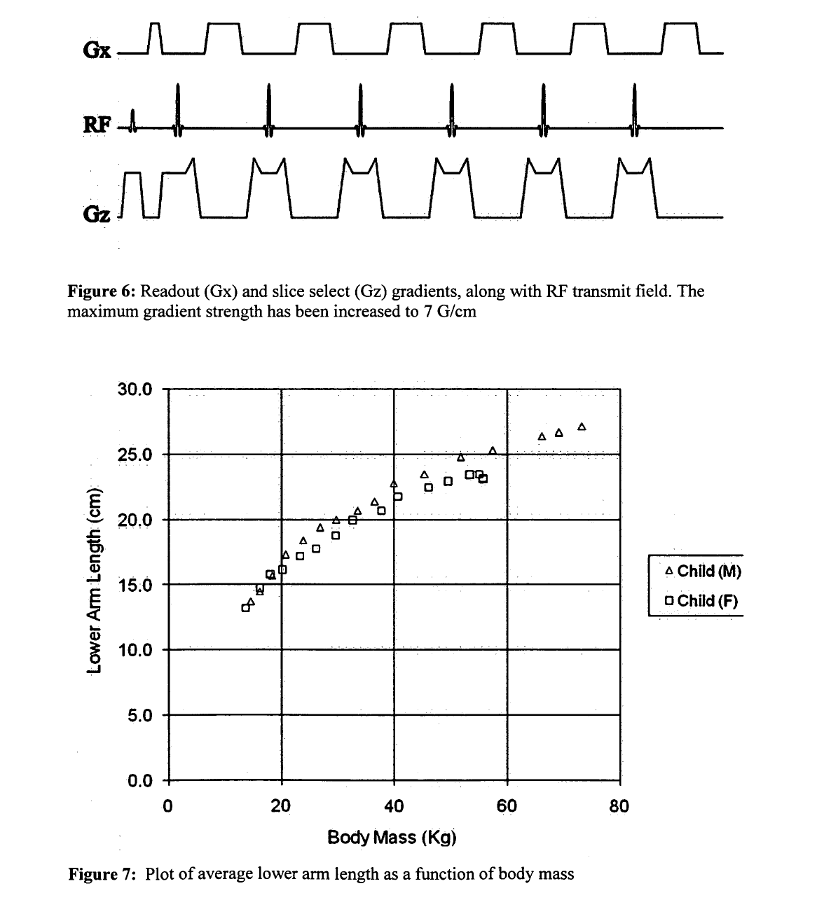

Compared to FIG. 2, FIG. 6 shows the improvement obtained by increasing the B₁ and the slice gradient in proportion. The total pulse length decreased dramatically, in this case by a factor of 2. The number of echoes was increased from 4 to 6, and the total fraction of time acquiring data therefore increased by 50%.

As is evident from these results, it is possible to increase the fraction of time spent acquiring data rather than manipulating spins by increasing the strength of both the gradient field and RF field during the excitation and the 180 degree spin rotations. This shortens the time needed for these pulses, allowing more time for the signal acquisition portion of the pulse sequence. By increasing both the gradient strength and B₁ in proportion, the frequency response of the pulse and therefore the slice profile is preserved. Therefore, we see that one cannot shorten these pulses without increasing both the gradient and RF field strengths without loss of slice profile. Thus, in a whole body system equipped with a localized RF transmit coil, gradient field limitations would limit the shortening of the 180 pulses. In a dedicated extremity or head system, the gradient fields can be ramped more quickly, and so the higher RF fields can also be used.

In addition to increased signal-to-noise, numerous other benefits are derived from changes that compress the time spent on spin manipulation. For example, blurring artifacts can be reduced in tissues with short T2 values (such as cartilage) by acquiring more data early in the decay, because more signal acquisition can occur with shorter 180 pulses. This acquisition strategy also reduces artifacts from magnetic susceptibility.

### Anatomical Specific Adjustment of Gradient Strength/Slew Rate and RF Fields

As explained in the previous section, the imaging performance can relate to the gradient strength, slew rate and B₁ transmit field strength. Making these quantities larger is desirable but is ultimately limited by fundamental physiological constraints. As described herein, the smaller diameter anatomical regions can safely tolerate higher B₁ and gradient strengths. In accordance with one embodiment of the invention, the SAR and/or PNS rates may be estimated, and the levels of B₁ and the gradient strength may be adjusted (up or down) on a pulse sequence, coil and anatomy specific model to optimize imaging performance while maintaining safe operation.

### Gradient Strength/Slew Rate Limitations as a Function of Scale Size

The Gradient strength and slew rate limitations, also expressed as dB/dt limits and electric field limits, have been well studied in the literature and are in integral part of the safety regulations such as IEC606010-2-33 [6]. Cardiac fibrillation is the primary concern in limiting the induced electric fields.

It has been shown that painful PNS always occurs below the Cardiac fibrillation threshold, so the safety standards allow PNS operation to levels of threshold of pain. As a practical matter, levels of operation below the pain threshold are required to prevent image artifacts due to patient discomfort and associated motion. By imaging just a portion of the body, such as the extremity, the concern about Cardiac fibrillation is greatly reduced but PNS must still be avoided for reasons of image quality.

To determine the benefits of imaging a smaller anatomy, such as the extremity, we turn to the basic scaling relationship between the electric and magnetic fields according to Faraday's law

$${\int }_{c} \vec{E} \cdot d \vec{l} = - \frac{d}{d t} {\int }_{S} \vec{B} \cdot d \vec{A} . • \qquad ( 1 )$$

If we scale all three dimensions simultaneously, keeping the time rate of magnetic field constant (same gradient performance), Equation 1 shows that the induced electric field reduces in proportion with the scale dimension. Therefore, if the gradient coil and anatomy dimensions are scaled down by a factor of 3 from a whole body diameter of 700 mm to a extremity sized coil of 230 mm, the resultant electric field is reduced by a factor 3. For a given ramp time, one can expect the onset of peripheral nerve stimulation to occur at a factor of 3 times higher gradient field strength.

A typical whole body gradient and RF coil with a 600 mm patient bore induces peripheral nerve stimulation in the range of 30 mT/m with rise times of 250 usec corresponding to slew rates of 120 T/m/sec. Testing of a small gradient system with a 180 mm diameter RF coil with large knee placed into the coil will all three gradient coils simultaneously operating at level of 70 mT/m and 200 T/m/sec slew rates resulted in no peripheral nerve stimulation. These results are consistent with this scaling.

Note also that Equation 1 is an integral over the anatomy showing that the electric fields are further reduced as the anatomy is smaller even if the coil is fixed size. As we can see from this analysis the smaller the anatomy and the smaller the gradient coil, the larger the values of gradient strength and slew rate before the onset of PNS.

### SAR Limitations as a Function of Scale Size

Given B₁ as the desired quantity, we would like to relate the heat energy deposited (SAR) into the patient as a function of system and anatomy scale size. Like gradient magnetic fields, the B₁ creates electric fields according to Faraday's law, however the gradient fields are at low frequency (0 to 20 KHz) and the concern is the induction of nerve stimulation. At the higher frequencies (64 MHz for 1.5T) of the B₁ magnetic fields, the concern is heating and therefore the scaling law is different.

In this case of SAR the power deposition is related to the square of the induced current density. The current density J in turn is related to the electric field according to Ohms law

\vec{J}=σ\vec{E} (2)

Hence for a given value of the applied B₁, by combining Equation 1 and 2, the local power deposition (SAR) will scale as the square of the scale size.

Table 1 summarizes the scaling laws for electric field and power deposition. Note that the scaling assumes all 3 dimensions scale in proportion. Variation from this scaling will occur due to anatomical difference but the general trend that smaller anatomies result in significantly lower SAR and induced electric fields remain.

TABLE 1
Scaling Laws

| Item | Units | Scaling Law |
|---|---|---|
| Electric Field | Volts/meter | r |
| Current Density | Amp/m\hat{ }2 | r |
| SAR | Watts/Kg | r² |
| (local power | | |
| deposition) | | |
| Total Energy | Watts | r⁵ |

For a typical whole body RF transmit coil, the maximum B₁ transmit field is in the range of 20 to 30 μT. Note that because of the safety aspect of SAR, the MRI safety standard IEC6061-2-33 [6] requires manufactures to list the maximum B₁ magnetic fields. The Philips Achieva documentation [7] for example shows a maximum B₁ of 27 μT for their whole body coil in what they consider to be “high” B₁ mode. The same document shows 20.25 uT as moderate and 13.5 uT is considered low. All manufactures have similar B₁ limits because the body itself is the primary limitation.

The above scaling laws show that as the coil and anatomy are reduced in size, the power deposition will reduce by the square of the size. As a result, for a size reduction of a factor of 3, it is possible to achieve a factor of 9 improvement in maximum B₁ magnetic fields. Also, by focusing energy on a part of the body, the local SAR is the applicable limit, and in the case of extremities, the local SAR is allowed to be higher, yielding an even higher value than would be predicted by scaling alone.

### Expression for SAR

As is demonstrated by the gradient and B₁ scaling above, a large benefit in performance is achievable if the maximum values are adjusted according to the size of the anatomy and/or specific RF or gradient coil while still maintaining operation inside safe limits. This is especially advantageous for imaging smaller anatomies since the structures are smaller, requiring higher performance to visualize. Fortunately, the scaling laws above show that the smaller anatomies are less coupled to the fields, and therefore higher transmit fields are permitted. Therefore, in accordance with one aspect of the invention, an anatomical model is used to estimate the size of the patient's anatomy for purposes of estimating the maximum tolerable B₁ or gradient magnet fields.

With an objective to estimate the maximum B₁ field at scan time, we refer to an expression that relates the B₁ and SAR for the particular anatomy and desired scan parameters. The computation is preferably efficient such that it can be used to optimize scan parameters in advance of scan without significant delay. There are a number variables that effect the local SAR including size and shape of tissue sample, the RF coil geometry as well as the specifics of the pulse sequence parameters. An exemplary modular method is described herein that reduces the SAR computation to its fundamental elements and allows coils and pulse sequences to be mixed and matched and used on all parts of the anatomy without having to characterize the matrix of all possible combinations.

As a starting point in the development of an expression for SAR, we consider the energy deposited in the patient for a single RF pulse. The energy is given by the integral

E=R_{S}∫(I(t)²dt, (3)

where R_{S} is the RF coil series loss contribution from the patient losses (excluding RF coil losses) and I/(t) is the RMS RF current as measured at the terminals of the RF coil. The series loss is largely independent of coil loading conditions and is therefore the quantity of interest.

For each transmit coil, the current and B₁ magnetic field are related by a scale factor according to:

I/(t)=λB₁(t), (4)

where λ is the amplitude of current required to produce a unity B₁ magnetic field.

Substitution of Equation 4 into Equation 3 yields

E=R_{S}λ²∫(B₁(t))²dt. (5)

We then sum over all RF pulses and arrive at the SAR for a single T/R (transmit and receive) period. The resultant SAR is given by:

$$S A R = q {λ}^{2} ( \frac{{R}_{S}}{M} ) ( \frac{1}{{T}_{R}} {\sum }_{i = 1}^{N} \int {( {B}_{1}^{i} ( t ) )}^{2} d t ) \frac{watts}{kg} \qquad ( 6 )$$

In (7), M is the mass of the tissue over which the energy is deposited, T_{R} is the T/R period in seconds and the summation is a sum over all of the RF pulses in a single T/R period. q is 1 for linear drive and 2 for quadrature (circularly polarized) transmit coils.

Equation 6 assumes that each TR period is representative time period for estimating SAR. It should be obvious that any suitable averaging time can be computed based on the details of the sequence.

Each of the parameters in Equation 6 is composed of information from various sources as summarized next. In some cases, such as with the RF coil characterization, the required information may be pre-calculated and saved in a configuration file. In other cases a computation may be done at scan time since important information such as patient anatomy or scan parameters are not known ahead of time:
λ This quantity is the amplitude of current required to produce a unity B₁ magnetic field. This quantity is experimentally or computational determined for each RF coil type and stored in a configuration file on the system. Also note that this quantity is the value from a single port of a coil while delivering power to multiple ports (for example, a quadrature coil).
R_{S} This quantity is the patient contribution to the coil series resistance in ohms. Note that the series resistance is chosen for the SAR computation because it is independent of coil tuning. For quadrature coils this resistance is the average of the two ports. This resistance is a function of the patient weight, anatomical landmark and resistance curve fit parameters as developed in a subsequent section of this disclosure.
q q=1 if the transmit coil is linear drive. q=2 if the transmit coil is a quadrature coil reflecting two ports dissipating power. This number can be stored in a configuration file and is specific to a particular coil design. Note that for quadrature coils, a lower value of λ will more offset the factor of two increase. The net reduction in SAR should ideally be 2 for perfect quadrature operation.
M This quantity is the mass in kg over which the RF power is to be averaged. For whole body SAR, M is the mass of the entire body. For local SAR, M is the local mass over which the RF energy is directly absorbed. The local mass is a function of the patient weight, landmark area and coil magnetic length as described later.

$$\frac{1}{{T}_{R}} {\sum }_{i = 1}^{N} \int {( {B}_{1}^{i} ( t ) )}^{2} d t$$

This quantity is the normalized energy loss for all scan RF pulses in a single T/R period. The details of the RF pulses used by any given sequence are known before the scan is run and these integrals can be quickly computed based on optimization of user scan parameter inputs.

### Estimating Size of the Anatomy

To develop a useful anatomical model, we wish to utilize minimum amount of patient information that is easily available but still achieve a reasonable estimate. It is generally possible to have patient weight and anatomical landmark (i.e., the part of the body being imaged), so we develop herein an exemplary model based on these two factors. Any inherent uncertainty or limitations by only using the weight information can be absorbed in the final safety margin so long as this is bounded and understood. Additional information such as height can be added to the model if necessary to reduce the uncertainly of the result if this becomes an important limitation. For purposes here, we illustrate this by developing a model for the upper and lower extremities.

### Exemplary Method of Analysis

A common reference cited is a book on biomechanics by DA Winter [8]. A table from the reference is reconstructed below (Table 2).

TABLE 2
Antropometric Data from DA Winter

| | | | Centre of Mass/ | | Radius of Gyration/ | | |
|---|---|---|---|---|---|---|---|
| | | Segment Wt/ | Segment Length | | Segment length | | |
| Segment | Definition | Total Body Wt | Proximal | Distal | C of G | Proximal | Distal |
| Hand | wrist/knuckle | 0.006 | 0.506 | 0.494 | 0.297 | 0.587 | 0.577 |
| | II digit 3 | | | | | | |
| Forearm | elbow/ | 0.016 | 0.43 | 0.57 | 0.303 | 0.526 | 0.647 |
| | ulnar | | | | | | |
| | styloid | | | | | | |
| Upper | G/H jt/ | 0.028 | 0.436 | 0.564 | 0.322 | 0.542 | 0.645 |
| arm | elbow | | | | | | |
| F'arm + | elbow/ | 0.022 | 0.682 | 0.318 | 0.468 | 0.827 | 0.565 |
| hand | ulnar | | | | | | |
| | styloid | | | | | | |
| Upper | G/H jt/ | 0.05 | 0.53 | 0.47 | 0.368 | 0.645 | 0.596 |
| limb | ulnar | | | | | | |
| | styloid | | | | | | |
| Foot | Lat. | 0.0145 | 0.5 | 0.5 | 0.475 | 0.69 | 0.69 |
| | mall/hd. | | | | | | |
| | MT2 | | | | | | |
| Shank | Fem. cond./ | 0.0465 | 0.433 | 0.567 | 0.302 | 0.528 | 0.643 |
| | med. Mall | | | | | | |
| Thigh | Gr. troch/ | 0.1 | 0.433 | 0.567 | 0.323 | 0.54 | 0.653 |
| | fem. cond. | | | | | | |
| Foot + | Fem. cond./ | 0.061 | 0.606 | 0.394 | 0.416 | 0.735 | 0.572 |
| shank | med. mall. | | | | | | |
| Lower | Gr. troch/ | 0.161 | 0.447 | 0.553 | 0.326 | 0.56 | 0.65 |
| Limb | med. mall. | | | | | | |

Data are from Winter DA (1979) “Biomechanics of human movement” Radius of Gyration/Segment length, p. 151

The information in Table 2 contains important anatomical ratios. However, to compute the mass of a portion of the joint over which the RF energy is deposited, we need this information along with an estimate of the length of the body segment.

As an example, consider the irradiated mass for imaging a segment of the lower arm. A reasonable estimate of the radiated mass could be obtained by assuming a conical shape to the lower arm. The shape of the cone is determined so center of mass is the same as that given by Winter (Table 2). We then integrate over the length of the arm surrounded by the coil to arrive at the mass. Similar expressions could be obtained for the knee, elbow leg etc. if the length of the upper arm and upper and lower leg (shank) were known. We can use the same information to obtain an average radius of the body section. This radius can be used to estimate the coupling of the RF or gradient fields to the body section.

To obtain a length estimate of the hand, foot, lower arm, upper arm, upper leg and lower leg, it is possible, for example, to analyze data from two primary sources. A first source may be data available on the Internet for children from the age of 2 to 19 provided by the U.S. government National Institutes and of Standards and Technology (NIST) [9]. A second source may be an anthropomorphic study of adult men and women conducted by the U.S. Army [10].

Ideally, one may analyze the raw data from the anthropometric studies allowing a direct estimate of the body segment length as a function of the weight, but the published results only show the information already statistically reduced to body weight and body segment length independently. Nevertheless, it is possible to make reasonable correlative estimates from the available data.

The underlying assumption in the analysis, is that size and weight are correlated. This is nothing more than a statement that heavier people are larger than lighter people which we know to be generally on average. For purposes here, the length of the body segment is to be correlated to weight by a power of the segment length. More specifically,

Lᵢ=αᵢM^{1/n}^{ᵢ} (7)

where Lᵢ is the length of the body segment i, M is the total mass of the body (not the body segment) and α_{i.} and nᵢ are values to be determined empirically for each segment.

If variations in human body sizes were to follow rules geometric scaling, i.e. if a heavier individual had all features in the same proportions as a lighter person, the scaling of weight would be with the cube of the size (n=3). We know by our own everyday experience that heavier people are not always taller, so we might expect the scaling to be less than cubic. It's worth noting that body mass index, used to determine if an individual is obese, underweight or overweight, is the ratio of weight/height squared. Not all heavier people are obese so we may expect the scaling to be greater than n=2. It turns out that an n value between 2 and 3 results in best for the fitting of anatomical dimensions outlined here. As shown herein, any n value in any suitable numerical increment between 2 and 3 may be employed, among others.

Now that a functional form for a relationship between body weight and segment size has been chosen, the next step is to determine the unknown coefficients for each body segment. The weight and segment length data provided for children are given as separate distributions, each as a function of age and sex.

Table 3 shows an example of the data available for the weight data of a male child [9]. Similar type data exists for each of the body segment lengths.

FIG. 7 is a plot of the mean lower arm length versus mean weight of the lower arm for males and female children, one data point for each age group. Note the minimal difference between the male and female results when the data is plotted this way indicating gender is not an important factor.

TABLE 3
Table 3: Example of data format for children from NIST.
WEIGHT KG. - (MALES)

| AGE/YRS | N | MEAN | S.D. | MIN | 5TH | 50TH | 95TH | MAX |
|---|---|---|---|---|---|---|---|---|
| 2.0-3.5 | 115 | 14.6 | 2.0 | 10.2 | 11.9 | 14.4 | 18.9 | 20.7 |
| 3.5-4.5 | 118 | 16.2 | 2.1 | 12.3 | 13.1 | 15.8 | 19.7 | 22.7 |
| 4.5-5.5 | 144 | 18.4 | 2.3 | 13.7 | 15.2 | 18.2 | 22.6 | 27.9 |
| 5.5-6.5 | 117 | 20.8 | 3.1 | 14.8 | 16.8 | 20.1 | 26.6 | 32.5 |
| 6.5-7.5 | 106 | 24.0 | 3.9 | 17.4 | 18.9 | 23.0 | 29.6 | 41.2 |
| 7.5-8.5 | 104 | 26.9 | 5.0 | 18.2 | 20.9 | 25.7 | 35.3 | 49.9 |
| 8.5-9.5 | 116 | 29.8 | 5.1 | 19.6 | 22.7 | 29.0 | 37.9 | 48.3 |
| 9.5-10.5 | 124 | 33.5 | 6.7 | 23.3 | 25.1 | 32.0 | 47.9 | 54.3 |
| 10.5-11.5 | 142 | 36.6 | 7.2 | 26.3 | 27.8 | 34.4 | 50.3 | 64.5 |
| 11.5-12.5 | 154 | 40.0 | 7.9 | 27.8 | 30.3 | 38.1 | 54.7 | 77.8 |
| 12.5-13.5 | 154 | 45.4 | 9.6 | 28.5 | 33.1 | 43.7 | 63.6 | 85.8 |
| 13.5-14.5 | 155 | 51.8 | 9.6 | 29.8 | 38.0 | 50.9 | 69.6 | 77.3 |
| 14.5-15.5 | 131 | 57.5 | 11.2 | 35.6 | 39.5 | 56.6 | 74.9 | 105.0 |
| 15.5-16.5 | 100 | 66.1 | 11.9 | 33.0 | 46.2 | 66.1 | 85.0 | 106.6 |
| 16.5-17.5 | 104 | 69.2 | 10.5 | 46.8 | 54.2 | 67.4 | 88.5 | 106.2 |
| 17.5-19.0 | 88 | 73.2 | 11.0 | 43.9 | 55.2 | 72.2 | 91.1 | 112.3 |

The adult Army data is not broken out according to age so we need a different approach to correlate the length and weight data. Table 4 is weight data from the army study presented in the form of percentiles. Inherent in Equation 7 relating weight and length is the assumption that length and weight increase together in a monotonic fashion. Therefore, to the extent that Equation 4 is true, we can then plot weight versus segment length for each percentile and expect a meaningful result.

TABLE 4
Format of Army data Weight

| Population | Females | Males |
|---|---|---|
| Percentile | kg | Kg |
| 1 | 45.24 | 55.27 |
| 2 | 47.02 | 57.83 |
| 3 | 48.13 | 59.43 |
| 5 | 49.64 | 61.59 |
| 10 | 51.98 | 64.93 |
| 15 | 53.61 | 67.22 |
| 20 | 54.94 | 69.8 |
| 25 | 56.12 | 70.71 |
| 30 | 57.2 | 72.21 |
| 35 | 58.24 | 73.61 |
| 40 | 59.24 | 74.98 |
| 45 | 60.24 | 76.33 |
| 50 | 61.25 | 77.69 |
| 55 | 62.29 | 79.7 |
| 60 | 63.37 | 80.5 |
| 65 | 64.51 | 82.02 |
| 70 | 65.75 | 83.64 |
| 75 | 67.13 | 85.45 |
| 80 | 68.72 | 87.51 |
| 85 | 70.62 | 89.96 |
| 90 | 73.11 | 93.16 |
| 95 | 76.98 | 98.7 |
| 97 | 79.59 | 101.35 |
| 98 | 81.56 | 103.8 |
| 99 | 84.7 | 107.71 |

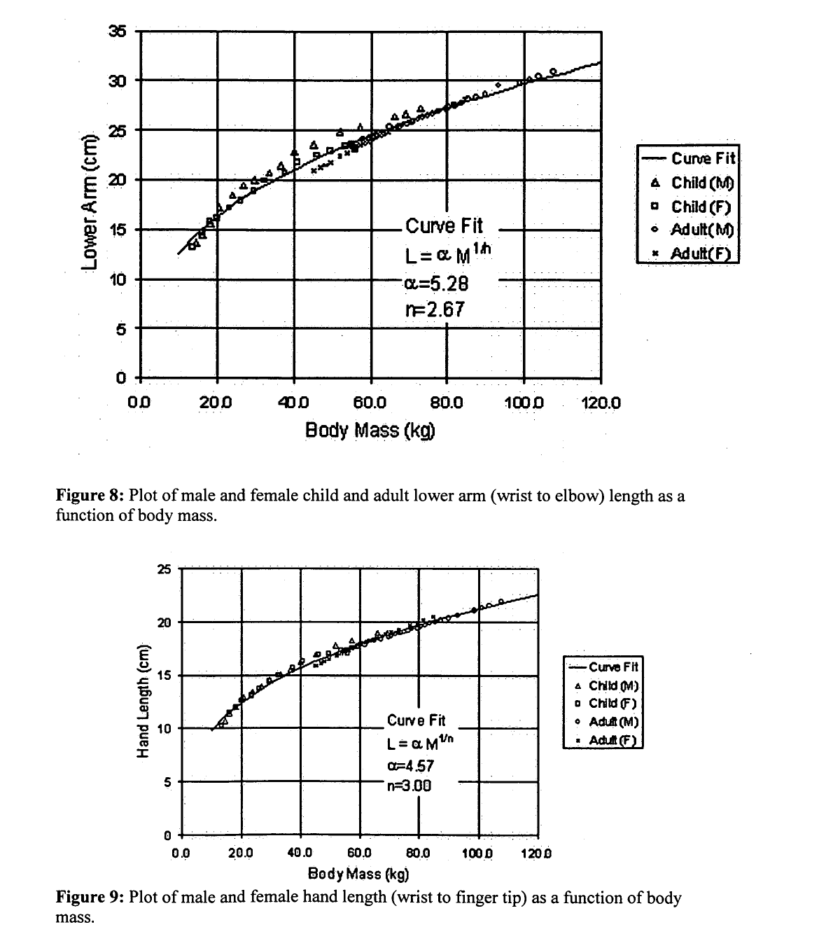

FIG. 8 combines the adult data to previously plotted children data. Also included is a single curve that represents a fit using Equation 7. The data fit was obtained by first taking the logarithm of the weight and length data and then performing a linear regression analysis on the result. The reason for the logarithm relates to the fact that any function of a single power is a straight line on a log/log plot. The ability to match the data points so well, confirms the validity of the form of Equation 7

The result shows remarkable consistency considering one study was conducted on children in the mid seventies and the other study on adults in the late eighties by completely separate groups. We are plotting averages against averages so the real error for any given person is larger than the scatter in the plot. An estimate of the error can be made as described below.

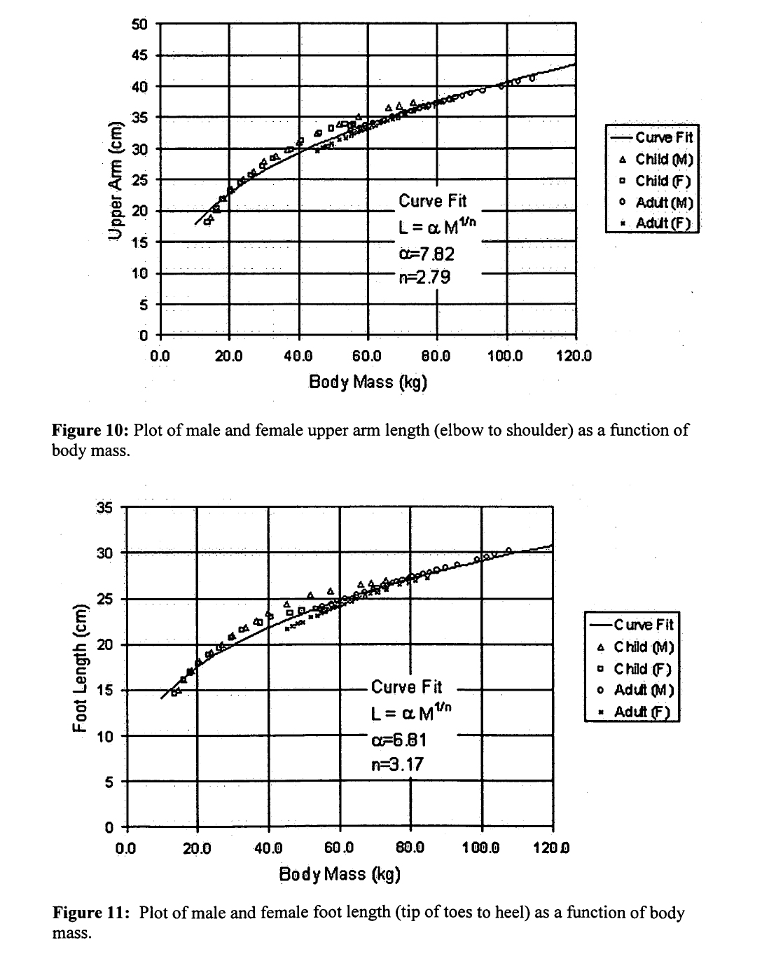

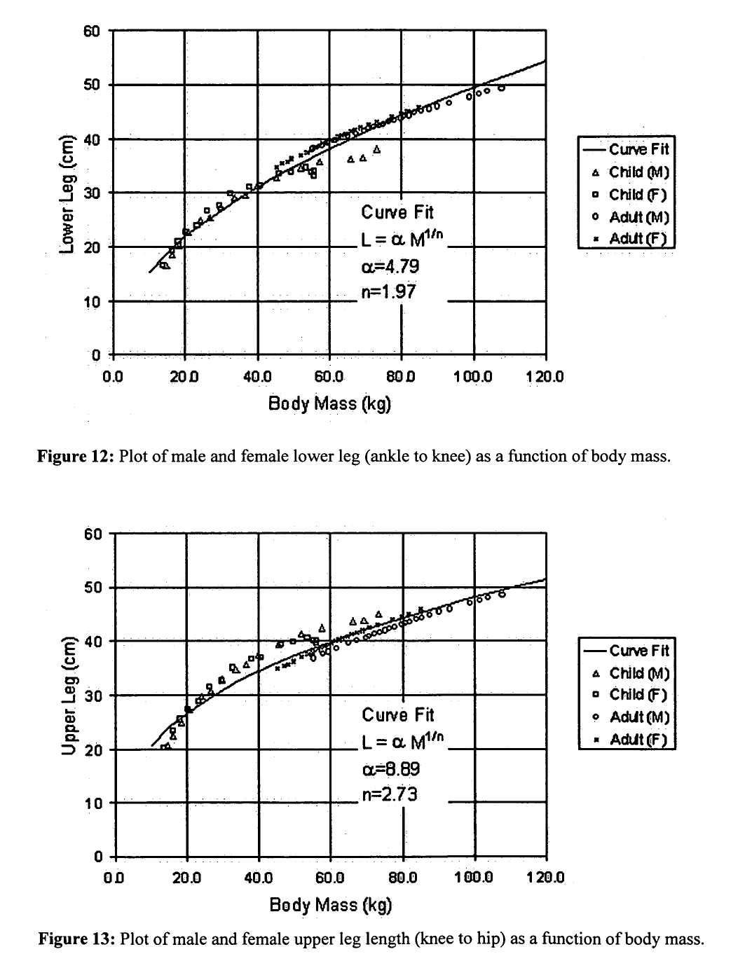

We now have a single formula that predicts an expected or average length of the forearm given the persons weight. FIGS. 9-13 show corresponding results for the hand, upper arm, foot, lower leg and upper leg segments respectively.

### Exemplary Estimate of Mass Exposed to the RF Power

For each of the eight landmark positions, a formula for estimating the mass exposed the RF power is now derived. An estimate is obtained by first approximating the lower arm, upper arm, lower leg, and upper leg by conical sections. The foot and the hand are approximated as cylinders. Depending on the landmark location, portions of the mass associated with adjacent body segment is added to arrive at the total irradiated mass.

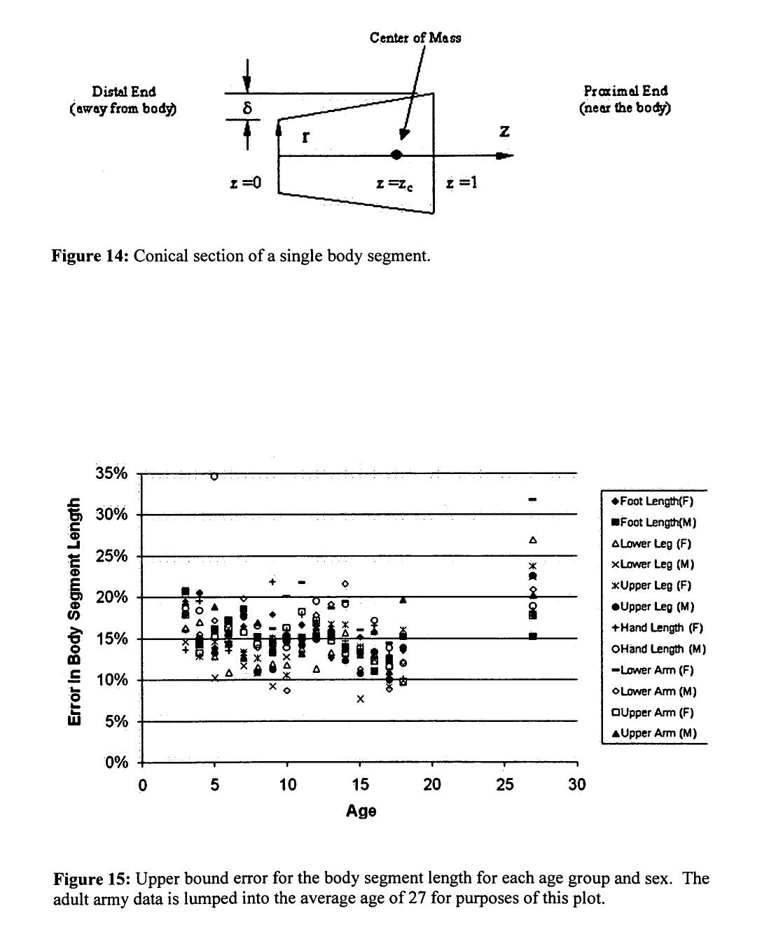

FIG. 14 below shows a conical section describing a single body segment. The location of the center of mass for each body segment is given by Winter [8] (Table 3). From Winter's center of mass data we determine the change in radius of the cone.

For purposes here we define a normalized cylindrical coordinate system. In this coordinate system the cone has a radius of 1 at the distal end and increases to 1+δ at the proximal end. Z is a coordinate that ranges from 0 to 1 over the length of the cone, again from distal to the proximal end.

The volume fraction of a portion of the cone is obtained by integrating over that portion and dividing by the total volume of the cone. The resultant fractional volume, V, is given by:

$$V ( δ , {z}_{1} , {z}_{2} ) = \frac{{z}_{2} ( 3 + 3 δ {z}_{2} + {( δ {z}_{2} )}^{2} ) - {z}_{1} ( 3 + 3 δ {z}_{1} + {( δ {z}_{1} )}^{2} )}{3 + 3 δ + {δ}^{2}} \qquad ( 8 )$$

wherein z₁ and z₂ are the integration limits in the z direction.

To relate the center of mass, z_{c} to the change in radius, δ, the assignment V(δ,0, z_{c})=½ is made to arrive at

$$\frac{{z}_{c} ( 3 + 3 δ {z}_{c} + {( δ {z}_{c} )}^{2} )}{3 + 3 δ + {δ}^{2}} = \frac{1}{2} \qquad ( 9 )$$

Equation 9 is then solved for δ to yield

$$δ = \frac{3 - 6 {z}_{c}^{2} - \sqrt{- 3 + 24 {z}_{c} - 36 {z}_{c}^{2} + 24 {z}_{c}^{3} - 12 {z}_{c}^{4}}}{- 2 + 4 {z}_{c}^{3}} \qquad ( 10 )$$

Table 2 by Winter shows a value of 0.57 for the center of mass for the lower arm. Substituting 0.57 into Equation 10 yields 0.333 for δ. This calculation is repeated for the upper arm and lower and upper leg center. The results are summarized in Table 5 along with the curve fits of segment length versus patient weight shown in FIGS. 8-13. The mass and lengths are functions of the total body mass, M. Each segment is a conical shape with an increase in diameter from Distal to Proximal end of the segment.

TABLE 5
Mass, Center of Mass and Length for body segments

| | | | Segment Diameter | | | | |
|---|---|---|---|---|---|---|---|
| | | | Increase from Distal to | | Segment Length | | |
| | Segment Mass | | Proximal End normalized to | | (cm) | | |
| Body | (kg) | | Segment Length | | L = α M^{1/n} | | |
| Segment | Symbol | Value | Symbol | Value | Symbol | δ | n |
| Hand | M_{H} | 0.006M | δ_{H} | 0 | L_{H} | 4.57 | 3.00 |
| Lower Arm | M_{LA} | 0.016M | δ_{LA} | 0.333 | L_{LA} | 5.28 | 2.67 |
| Upper Arm | M_{UA} | 0.028M | δ_{UA} | 0.299 | L_{UA} | 7.82 | 2.79 |
| Foot | M_{F} | 0.0145M | δ_{F} | 0 | L_{F} | 6.81 | 3.17 |
| Lower Leg | M_{LL} | 0.0465M | δ_{LL} | 0.316 | L_{LL} | 4.79 | 1.97 |
| Upper Leg | M_{UL} | 0.1M | δ_{UL} | 0.316 | L_{UL} | 8.89 | 2.73 |

Using the mass of the body segment, the length of the body segment and a weighted sum of the volumes from each segment, a formula for the irradiated mass is developed specific to each landmark area. If the RF coil encompasses the entire segment of interest, the entire mass of the segment is used with an additional contribution form the neighboring body segments.

The resultant formulae are summarized in Table 6 for each of the anatomical areas. Following are some of the factors considered when developing these formulas.
Hand: The hand is in the axial center of the coil. A small hand of a child is likely to be smaller than the magnetically active length of the RF coil. Most adults with a special hand coil will probably have a hand larger than the length of the coil. This situation is covered by testing for this condition and calculating the mass accordingly. If the coil is longer than the hand, a portion of the lower arm is included in the mass calculation
Wrist: The wrist is in the axial center of the coil. If the “half length” of the coil is less than the length of the hand, a contribution to the irradiated mass from the hand and lower arm is required. If the coil half length is longer than the hand, the irradiated mass is the entire mass of the hand plus a contribution from the lower arm.
Lower Arm: The lower arm is centered axially in the coil. Three cases are considered. One case involves only the lower arm for a sufficiently short coil. The second case involves a portion of the hand, the entire lower arm and a portion of the upper arm. The third case (unlikely to be encountered except in small children) involves the entire hand, the entire lower arm and a portion of the upper arm.
Elbow: The elbow in the axial center of the coil. If the half length of the coil is less than the length of the lower arm, the upper and lower arms both contribute to the mass. If the half length of the coil is greater the length of the lower arm (an unlikely situation) the entire mass of the lower arm may be used but the mass of the hand may be ignored (conservative estimate).
Foot: Treated in a manner analogous to the hand.
Ankle: Treated in a manner analogous to the wrist. Due to the shape of the ankle and foot, this will yield a slightly under estimate of the mass giving a slightly conservative estimate of SAW
Lower Leg: Treated in a manner analogous to the lower arm.
Knee: Treated in a manner analogous to the elbow.
   Table 6 summarizes the irradiated mass for each of eight anatomical locations (also called landmarks) that would normally be used as landmark position in the scanner. The irradiated mass is a function of the RF coil length, L_{C}, the masses of the six body segments and a volume fraction of the each body segment designated by V. V is calculated using Equation 8.

TABLE 6
Irradiated Mass Formulae for Particular Anatomical Choice.

| Anatomy | | |
|---|---|---|
| (landmark) | Irradiated Mass | Test Case |
| Hand | ${M}_{H} V ( {δ}_{H} , \frac{{L}_{H} - {L}_{C}}{2 {L}_{H}} , \frac{{L}_{H} + {L}_{C}}{2 {L}_{H}} )$ | L_{C} < L_{H} |
| | ${M}_{H} + {M}_{LA} V ( {δ}_{LA} , 0 , \frac{( {L}_{C} - {L}_{H} )}{2 {L}_{LA}} )$ | L_{C} ≧ L_{H} |
| Wrist | ${M}_{H} V ( {δ}_{H} , 1 - \frac{{L}_{C}}{2 {L}_{H}} , 1 ) + {M}_{LA} V ( {δ}_{LA} , 0 , \frac{{L}_{C}}{2 {L}_{LA}} )$ | L_{C} < 2L_{H} |
| | ${M}_{H} + {M}_{LA} V ( {δ}_{LA} , 0 , \frac{{L}_{C}}{2 {L}_{LA}} )$ | L_{C} ≧ 2L_{H} |
| Arm | ${M}_{LA} V ( {δ}_{LA} , \frac{{L}_{LA} - {L}_{C}}{2 {L}_{LA}} , \frac{{L}_{LA} + {L}_{C}}{2 {L}_{LA}} )$ | L_{C} < L_{LA} |
| | ${M}_{LA} + {M}_{H} V ( {δ}_{H} , 1 - \frac{{L}_{C} - {L}_{LA}}{2 {L}_{H}} , 1 ) + {M}_{UA} V ( {δ}_{UA} , 0 , \frac{{L}_{C} - {L}_{LA}}{2 {L}_{UA}} )$ | L_{LA} ≦ L_{C} < (L_{LA} + 2L_{H}) |
| | ${M}_{LA} + {M}_{H} + {M}_{UA} V ( {δ}_{UA} , 0 , \frac{{L}_{C} - {L}_{LA}}{2 {L}_{UA}} )$ | L_{C} ≧ (L_{LA} + 2L_{H}) |
| Elbow | ${M}_{LA} V ( {δ}_{LA} , 1 - \frac{{L}_{C}}{2 {L}_{LA}} , 1 ) + {M}_{UA} V ( {δ}_{UA} , 0 , \frac{{L}_{C}}{2 {L}_{UA}} )$ | L_{C} < (L_{LA} + L_{UA}) |
| | M_{LA} + M_{UA} | L_{C} ≧ (L_{LA} + L_{UA}) |
| Foot | ${M}_{F} V ( {δ}_{F} , \frac{{L}_{F} - {L}_{C}}{2 {L}_{F}} , \frac{{L}_{F} + {L}_{C}}{2 {L}_{F}} )$ | L_{C} < L_{F} |
| | ${M}_{F} + {M}_{LL} V ( {δ}_{LL} , 0 , \frac{( {L}_{C} - {L}_{F} )}{2 {L}_{LL}} )$ | L_{C} ≧ L_{F} |
| Ankle | ${M}_{F} V ( {δ}_{F} , 1 - \frac{{L}_{C}}{2 {L}_{F}} , 1 ) + {M}_{LL} V ( {δ}_{LL} , 0 , \frac{{L}_{C}}{2 {L}_{LL}} )$ | L_{C} < 2L_{F} |
| | ${M}_{F} + {M}_{LL} V ( {δ}_{LL} , 0 , \frac{{L}_{C}}{2 {L}_{LL}} )$ | L_{C} ≧ 2L_{F} |
| Leg | ${M}_{LL} V ( {δ}_{LL} , \frac{{L}_{LL} - {L}_{C}}{2 {L}_{LL}} , \frac{{L}_{LL} + {L}_{C}}{2 {L}_{LL}} )$ | L_{C} < L_{LL} |
| | ${M}_{LL} + {M}_{F} V ( {δ}_{F} , 1 - \frac{{L}_{C} - {L}_{LL}}{2 {L}_{F}} , 1 ) + {M}_{UL} V ( {δ}_{UL} , 0 , \frac{{L}_{C} - {L}_{LL}}{2 {L}_{UL}} )$ | L_{LL} ≦ L_{C} < (L_{LL} + 2L_{F}) |
| | ${M}_{LL} + {M}_{F} + {M}_{UL} V ( {δ}_{UL} , 0 , \frac{{L}_{C} - {L}_{LL}}{2 {L}_{UL}} )$ | L_{C} ≧ (L_{LL} + 2L_{F}) |
| Knee | ${M}_{LL} V ( {δ}_{LL} , 1 - \frac{{L}_{C}}{2 {L}_{LL}} , 1 ) + {M}_{UL} V ( {δ}_{UL} , 0 , \frac{{L}_{C}}{2 {L}_{UL}} )$ | L_{C} < (L_{LL} + L_{UL}) |
| | M_{LL} + M_{UL} | L_{C} ≧ (L_{LL} + L_{UL}) |

### The Uncertainty in the Irradiated Mass

As displayed in Table 5 and Table 6, the irradiated mass is determined from the patient total body mass. From the total body mass two primary anatomical factors are calculated, an estimate of the segment length and an estimate of the segment mass. Any remaining error in our estimate can be thought of as an error in either the anatomical weight or length.

If we examine a sufficiently short section of the body segment, the irradiated mass has a functional form of

$$Irradiated Mass = \frac{{M}_{BS}}{{L}_{BS}} {L}_{C} \qquad ( 11 )$$

where M_{BS} is the mass of the body segment, L_{BS}, is the length of the body segment and L_{C} is the length of the coil. Expanding Equation 11 about small changes in the body mass and small changes in the length yields

$$Irradiated Mass = \frac{{M}_{BS}}{{L}_{BS}} {L}_{C} [ 1 + \frac{Δ {M}_{BS}}{{M}_{BS}} - \frac{Δ {L}_{BS}}{{L}_{BS}} + … ] \qquad ( 12 )$$

where Δ is used to refer to a small change in the segment mass or length.

Equation 12 shows that the fractional error in the segment mass and the fractional error in the segment length each contribute proportionally to the error in the irradiated mass. The fractional error in the segment length is the greater source of error for reasons explained next.

Consider the fact that the body mass is the total mass of the soft tissue plus skeletal structure. On the other hand, the body segment length is the length of the skeletal structure, but does not factor the amount of tissue on that structure. Common every day experience tells us that there is a wide variation in weight for the same skeletal lengths. However, if we compare a lighter person to a heavier person with the same skeletal lengths we expect the extra mass of the heavier person to be disturbed evenly throughout the body. Therefore, the error in the segment length is the most important element in estimating the error in the irradiated mass.

The child data is given in 1 year age intervals (except the 1^{st} and last group covering a 1.5 year span) along with a maximum and minimum body segment length for each age interval. Likewise, the Army adult data gives a maximum and minimum value of length for each body segment. Lets now define the fractional error in body segment length to be

$$Body Segment Length Error ( \% ) = \frac{{L}_{BS}^{max} - {L}_{BS}^{min}}{2 {L}_{BS}^{avg}} \qquad ( 13 )$$

This error can be considered a worst case since it represents the difference between two people at the extreme upper and lower limit in size in each age category without regard to any correlation with weight.

FIG. 15 is a plot of the error for each child age group as well as the US Army adult data. There is an anomalous data point for the hand data of a 5 year old male child. This data is the result of a child with an extremely small hand (5.5 cm) or is an error in transcribing the data.

Overall, we see that the upper bound in the fractional error in body segment length is clustered in the 10% to 20% range with an extreme maximum error approaching 30%. Given the large sample set, over 2000 persons in the Army study and approximately 2000 male children and 2000 female children spread out in age fairly uniformly, the range of variation is sampled well.

A review of the standard deviation of the segment lengths in a couple of cases and found that it typical value was in the 5 to 10% range, considerably less than the errors displayed. For example, the standard deviation of the lower arm for the Army study about 6% of the average length of the lower arm. The 32% error displayed in FIG. 15 is more than 5 standard deviations from the average. FIG. 15 therefore represents the extreme error.

We now estimate the limb radius based on patient weight anatomical landmark. Rather than creating estimates of the limb radius directly, the average radius of the limb over the length of the coil can be determined from a prior estimate of the irradiated mass and conservation of mass. The radius is then given by the simple formula

$$r = \sqrt{\frac{1000 M}{π {L}_{C} ρ}} \qquad ( 14 )$$

where M is the irradiated mass in kg, L_{C} is the magnetic length the coil in cm and ρ is the average body mass density in grams per cubic cm. Note that L_{C} is the same coil length used in the computation of the irradiated mass.

### Series Resistance Calculation

We now show a methodology for estimating the RF coil series resistance from body mass and anatomical landmark. For Extremities, it is highly desirable to derive a single expression for the coil series resistance in terms of the average radius of the limb section that is applicable to all parts of the limbs, i.e. wrist arm, knee, leg, hand.

For a given applied RF magnetic field, the electric field and hence the RF currents in the sample increase in proportion to the distance from the center of the sample. If all dimensions scale in proportion, the total energy dissipated scales as the product of the volume (3^{rd} power with dimension) times the local power dissipation (square of the dimension) for a 5^{th} power scaling law as shown earlier in Table 1. If we integrate over a long cylindrical sample, volume increases as the square of the radius so the total energy in the sample should scale as the 4^{th} power of the sample radius. It therefore seems reasonable that the controlling variable for the sample resistance should be the radius of the limb and not the type of limb. For example, the leg of a child should have approximately the same dissipation as the arm of an adult as long as the two are of the same radius.

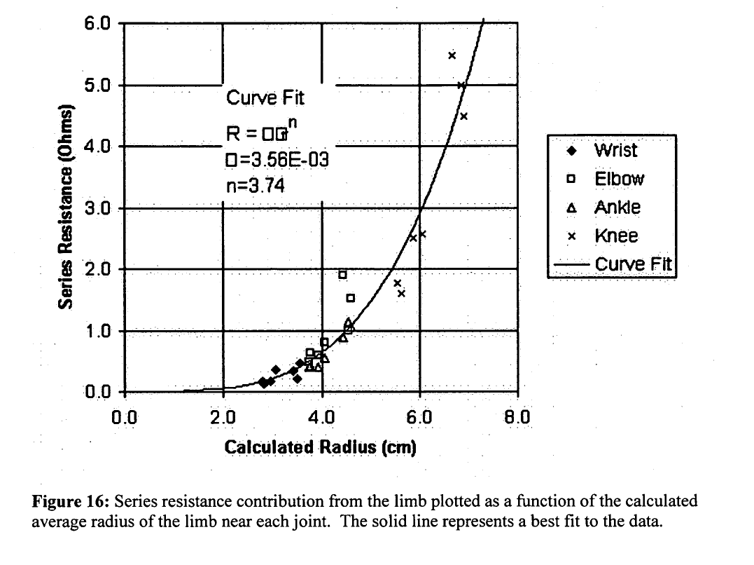

To demonstrate the validity of this approach, the RF coil series resistance was measured and plotted as a function of the radius computed from Equation 14 for a number of the anatomical landmarks. The results are given in FIG. 16. Note the resultant curve fit scales as the 3.74 power, in reasonable agreement with the 4^{th} power law.

### B₁ and Gradient Adjustment During Scan Setup

During Scan setup an estimate of SAR is made using the methods described here. First the patient weight and anatomy landmark position is determined by operator input. An estimate of the irradiated mass the then made using the patient weight, anatomy, coil length, and the equations in Table 5 and Table 6. Equation 14 is used to estimate the radius from which the series resistance is computed using Equation 7. Integrals of the B₁ magnetic field are then made. SAR is then estimated using Equation 6. If the SAR is below operating limits, the B₁ magnetic field, and gradient strengths are increased resulting in a closer packed echo train as in 6.

All statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Block diagrams and other representations and descriptions of system components and circuitry herein represent conceptual views of illustrative circuitry and software embodying the principles of the invention. Thus the functions of the various elements shown in the Figures and described in the text hereof may be provided through the use of dedicated hardware as well as hardware capable of executing software in association with appropriate software. When provided by a controller and/or a processor, the functions may be provided by a single dedicated processor, by a single shared processor, or by a plurality of individual processors, some of which may be shared. The functions of those various elements may be implemented by, for example, digital signal processor (DSP) hardware, network processor, application specific integrated circuit (ASIC), field programmable gate array (FPGA), read-only memory (ROM) for storing software, random access memory (RAM), and non-volatile storage. Other hardware, conventional and/or custom, may also be included.

In the claims hereof any element expressed as a means for performing a specified function is intended to encompass any way of performing that function including, for example, a) a combination of circuit elements which performs that function or b) software in any form, including, therefore, firmware, microcode or the like, combined with appropriate circuitry for executing that software to perform the function. The invention as defined by such claims resides in the fact that the functionalities provided by the various recited means are combined and brought together in the manner which the claims call for. Applicants thus regard any means which can provide those functionalities as equivalent to those shown herein.

Similarly, it will be appreciated that the system flows described herein represent various processes which may be substantially represented in computer-readable medium and so executed by a computer or processor, whether or not such computer or processor is explicitly shown. Moreover, the various processes can be understood as representing not only processing and/or other functions but, alternatively, as blocks of program code that carry out such processing or functions.

It will be apparent to those skilled in the art that various modifications and variations can be made in the devices and methods of the present invention without departing from the spirit or scope of the invention. Thus, it is intended that the present invention include modifications and variations that are within the scope of the subject disclosure and equivalents.

### REFERENCES

Each of the below references is incorporated by reference herein in its entirety):
[1] http://www.esaote.com/products/MRI/products1.htm
[2] www.onicorp.com
[3] 4,825,162 Nuclear magnetic resonance (NMR) imaging with multiple surface coils.
[4] Roemer P B, Edelstein W A, Hayes C E, Souza S P, and Mueller O M, “The NMR Phased-Array.” Magnetic Resonance in Medicine 16, p. 192-225 (1990).
[5] Sodickson, D. K., and Manning, W. J., “Simultaneous acquisition of spatial harmonics (SMASH): Fast imaging with radiofrequency coil arrays.” Magnetic Resonance in Medicine 38: 591-603.
[6] “Medical electrical equipment Particular requirements for the safety of magnetic resonance equipment for medical diagnosis”, International Standard IEC 60601-2-33.
[7] “Philips Acheiva Release 1.2 Technical Description” (2004).
[8] Winter DA “Biomechanics of human movement” (1979).
[9] http://www.nist.gov/itl/div894/ovrt/projects/anthrokids/content.htm
[10] “1988 Anthropometric Survey of US Army Personnel: Methods and Summary Statistics”, US Army technical report NATIC/KTR-89/044, September 1989.

1. A system for performing imaging comprising a MR scanner, the MR scanner including:
a) at least one local radio-frequency transmit coil;
b) at least one local gradient coil, wherein the at least one local radio-frequency transmit coil and at least one local gradient coil cooperate to define an imaging volume;
c) a control system for performing an imaging operation on a patient's anatomy disposed within the imaging volume, wherein the control system permits a selective simultaneous increase of the gradient magnetic field strength and peak B₁ field strength by substantially the same factor, f.

2. The system of claim 1, wherein the control system reduces the duration of the B₁ transmit pulse by the factor, f when the control system increases the gradient magnetic field strength and peak B₁ field strength by the factor, f.

3. The system of claim 1, wherein the frequency response and the spatial response of the excited spins of the region being imaged is substantially preserved as a result of simultaneously increasing the gradient magnetic field strength and peak B₁ field strength in substantially the same proportion.

4. The system of claim 1, wherein the flip angle resulting from the transmit pulse is substantially unchanged when increasing the gradient and peak B₁ field strength.

5. The system of claim 1, wherein the excitation properties of the combined RF transmit and gradient pulse are preserved when increasing the gradient and peak B₁ field strength.

6. The system of claim 1, wherein the control system is adapted and configured to increase the gradient strength in excess of 30 mT/m.

7. The system of claim 1, wherein the control system is adapted and configured to increase the gradient strength at a rate in excess of 100 T/m/sec.

8. The system of claim 1, wherein the control system is adapted and configured to create an estimate of a physiological limit of a region to be imaged.

9. The system of claim 8, wherein the estimate is based at least in part on a predetermined value.

10. The system of claim 9, wherein the estimate is based at least in part on the weight of a patient.

11. The system of claim 10, wherein the estimate is based at least in part on a predetermined value established for the particular anatomy being imaged.

12. The system of claim 8, wherein an estimate of the mass to be imaged is created based on one or more factors selected from the group consisting of (i) the weight of the patient, (ii) the anatomy being imaged, and (iii) the size of the imaging coil.

13. The system of claim 8, wherein the factor f is maximized for a given physiological limit.

14. The system of claim 8, wherein the physiological limit relates to SAR arising from application of the B₁ field.

15. The system of claim 1, wherein the B₁ field is maximized to not exceed a limit of 20 Watts/kg.

16. The system of claim 8, wherein the physiological limit relates to peripheral nerve stimulation arising from electric fields induced in the patient's anatomy.

17. The system of claim 1, wherein the control system is adapted and configured to increase the ramp rate of the gradient field.

18. The system of claim 1, wherein the control system is adapted and configured to reduce the duration of the echo train.

19. The system of claim 18, wherein the echo train can be reduced by about 25 percent by the control system.

20. The system of claim 18, wherein the imaging sequence is optimized for imaging tissues with relatively short T2 values.

21. The system of claim 20, wherein the imaging sequence is optimized for cartilage.

22. The system of claim 1, wherein the control system is adapted and configured to selectively increase the duration of the data acquisition window for an echo train of a predetermined duration in cooperation with simultaneously increasing the gradient magnetic field strength and peak B₁ field strength.

23. The system of claim 1, wherein the gradient field strength may be selectively increased by the control system to a magnitude between about 5 G/cm and about 10 G/cm.

24. The system of claim 1, wherein the duration of the B₁ pulse is decreased to between about 0.5 msec and about 1.0 msec.

25. The system of claim 1, wherein the magnitude of the maximum B₁ field strength is more than about 0.4 gauss.

26. The system of claim 1, wherein the signal to noise ratio increases by about 25% as a result of simultaneously increasing the gradient magnetic field strength and peak B₁ field strength.

27.-255. (canceled)
